SEQUENCE LISTING (48 sequences)

SEQ ID NO: 1 - protein, 10 aa, ARTIFICIAL SEQUENCE
  HYD1 sequence
KIKMVISWKG

SEQ ID NO: 2 - protein, 10 aa, ARTIFICIAL SEQUENCE
  HYD1 variant
AIAMVISWAG

SEQ ID NO: 3 - protein, 10 aa, ARTIFICIAL SEQUENCE
  HYD1 variant
AIKMVISWAG

SEQ ID NO: 4 - protein, 10 aa, ARTIFICIAL SEQUENCE
  HYD1 variant
AIKMVISWKG

SEQ ID NO: 5 - protein, 7 aa, ARTIFICIAL SEQUENCE
  HYD1 variant
AKMVISW

SEQ ID NO: 6 - protein, 9 aa, ARTIFICIAL SEQUENCE
  HYD1 variant
AKMVISWKG

SEQ ID NO: 7 - protein, 7 aa, ARTIFICIAL SEQUENCE
  HYD1 variant
IAMVISW

SEQ ID NO: 8 - protein, 9 aa, ARTIFICIAL SEQUENCE
  HYD1 variant
IAMVISWKG

SEQ ID NO: 9 - protein, 7 aa, ARTIFICIAL SEQUENCE
  HYD1 variant
IKAVISW

SEQ ID NO: 10 - protein, 9 aa, ARTIFICIAL SEQUENCE
  HYD1 variant
IKAVISWKG

SEQ ID NO: 11 - protein, 7 aa, ARTIFICIAL SEQUENCE
  HYD1 variant
IKMAISW

SEQ ID NO: 12 - protein, 9 aa, ARTIFICIAL SEQUENCE
  HYD1 variant
IKMAISWKG

SEQ ID NO: 13 - protein, 7 aa, ARTIFICIAL SEQUENCE
  HYD1 variant
IKMVASW

SEQ ID NO: 14 - protein, 9 aa, ARTIFICIAL SEQUENCE
  HYD1 variant
IKMVASWKG

SEQ ID NO: 15 - protein, 7 aa, ARTIFICIAL SEQUENCE
  HYD1 variant
IKMVIAW

SEQ ID NO: 16 - protein, 9 aa, ARTIFICIAL SEQUENCE
  HYD1 variant
IKMVIAWKG

SEQ ID NO: 17 - protein, 7 aa, ARTIFICIAL SEQUENCE
  HYD1 variant
IKMVISA

SEQ ID NO: 18 - protein, 9 aa, ARTIFICIAL SEQUENCE
  HYD1 variant
IKMVISAKG

SEQ ID NO: 19 - protein, 7 aa, ARTIFICIAL SEQUENCE
  HYD1 variant
IKMVISW

SEQ ID NO: 20 - protein, 9 aa, ARTIFICIAL SEQUENCE
  HYD1 variant
IKMVISWAG

SEQ ID NO: 21 - protein, 8 aa, ARTIFICIAL SEQUENCE
  HYD1 variant
KMVISWKA

SEQ ID NO: 22 - protein, 9 aa, ARTIFICIAL SEQUENCE
  HYD1 variant
IKMVISWKG

SEQ ID NO: 23 - protein, 5 aa, ARTIFICIAL SEQUENCE
  HYD1 variant
ISWKG

SEQ ID NO: 24 - protein, 10 aa, ARTIFICIAL SEQUENCE
  HYD1 variant
KAKMVISWKG

SEQ ID NO: 25 - protein, 10 aa, ARTIFICIAL SEQUENCE
  HYD1 variant
KIAMVISWAG

SEQ ID NO: 26 - protein, 10 aa, ARTIFICIAL SEQUENCE
  HYD1 variant
KIAMVISWKG

SEQ ID NO: 27 - protein, 10 aa, ARTIFICIAL SEQUENCE
  HYD1 variant
KIKAVISWKG

SEQ ID NO: 28 - protein, 10 aa, ARTIFICIAL SEQUENCE
  HYD1 variant
KIKMAISWKG

SEQ ID NO: 29 - protein, 5 aa, ARTIFICIAL SEQUENCE
  HYD1 variant
KIKMV

SEQ ID NO: 30 - protein, 10 aa, ARTIFICIAL SEQUENCE
  HYD1 variant
KIKMVASWKG

SEQ ID NO: 31 - protein, 6 aa, ARTIFICIAL SEQUENCE
  HYD1 variant
KIKMVI

SEQ ID NO: 32 - protein, 10 aa, ARTIFICIAL SEQUENCE
  HYD1 variant
KIKMVIAWKG

SEQ ID NO: 33 - protein, 7 aa, ARTIFICIAL SEQUENCE
  HYD1 variant
KIKMVIS

SEQ ID NO: 34 - protein, 10 aa, ARTIFICIAL SEQUENCE
  HYD1 variant
KIKMVISAKG

SEQ ID NO: 35 - protein, 8 aa, ARTIFICIAL SEQUENCE
  HYD1 variant
KIKMVISW

SEQ ID NO: 36 - protein, 10 aa, ARTIFICIAL SEQUENCE
  HYD1 variant
KIKMVISWAG

SEQ ID NO: 37 - protein, 9 aa, ARTIFICIAL SEQUENCE
  HYD1 variant
KIKMVISWK

SEQ ID NO: 38 - protein, 10 aa, ARTIFICIAL SEQUENCE
  HYD1 variant
KIKMVISWKA

SEQ ID NO: 39 - protein, 8 aa, ARTIFICIAL SEQUENCE
  HYD1 variant
KMVISWKG

SEQ ID NO: 40 - protein, 5 aa, ARTIFICIAL SEQUENCE
  HYD1 variant
LSWKG

SEQ ID NO: 41 - protein, 7 aa, ARTIFICIAL SEQUENCE
  HYD1 variant
MVISWKG

SEQ ID NO: 42 - protein, 4 aa, ARTIFICIAL SEQUENCE
  HYD1 variant
SWKG

SEQ ID NO: 43 - protein, 6 aa, ARTIFICIAL SEQUENCE
  HYD1 variant
VISWKG

SEQ ID NO: 44 - protein, 10 aa, ARTIFICIAL SEQUENCE
  HYD1 variant
WIKSMKIVKG

SEQ ID NO: 45 - protein, 6 aa, ARTIFICIAL SEQUENCE
  HYD1 variant
KMVIXW

SEQ ID NO: 46 - protein, 9 aa, ARTIFICIAL SEQUENCE
  HYD1 variant
IKMVISWXX

SEQ ID NO: 47 - protein, 8 aa, ARTIFICIAL SEQUENCE
  HYD1 variant
KMVISWXX

SEQ ID NO: 48 - protein, 9 aa, ARTIFICIAL SEQUENCE
  HYD1 variant
XKMVISWXX

(19) United States
(12) Patent Application Publication
(10) Pub. No.: US 2014/0080762 A1
(43) Pub. Date: Mar. 20, 2014

(54) HYD1 PEPTIDES FOR RELAPSED CANCER

(71) Applicants: Lori Anne Hazlehurst, Ruskin, FL (US); William S. Dalton, Temple Terrace, FL (US); Michael Emmons, Tampa, FL (US); Anthony W. Gebhard, St. Petersburg, FL (US)

(72) Inventors: Lori Anne Hazlehurst, Ruskin, FL (US); William S. Dalton, Temple Terrace, FL (US); Michael Emmons, Tampa, FL (US); Anthony W. Gebhard, St. Petersburg, FL (US)

(73) Assignees: H. LEE MOFFITT CANCER CENTER AND RESEARCH INSTITUTE, INC., TAMPA, FL (US); UNIVERSITY OF SOUTH FLORIDA, TAMPA, FL (US)

(21) Appl. No.: 14/006,602

(22) PCT Filed: Mar. 21, 2012
(86) PCT No.: PCT/US2012/029993

§ 371 Date: Sep. 20, 2013

Related U.S. Application Data
(60) Provisional application No. 61/454,919, filed on Mar. 21, 2011.

(51) Int. Cl. A61K 38/08; C07K 7/06; G01N 33/50; A61K 45/06

(52) U.S. Cl. CPC A61K 38/08; A61K 45/06; C07K 7/06; G01N 33/5011
USPC 514/19.1; 514/19.3; 530/389.7; 435/375; 435/7.1; 435/325; 506/18; 506/16

(57) ABSTRACT

The subject invention pertains to compositions and methods for treatment of malignancies and inhibiting the growth of cancer cells, such as multiple myeloma and other hematologic malignancies, using HYD1 peptides. Other aspects of the invention are directed to methods for selection of agents useful in the treatment of malignancies and inhibiting the growth of cancer cells. Further aspects of the invention include methods for determining whether a cancer is sensitive or resistant to treatment with HYD1 peptides based on the presence of certain biomarkers, such as α4 integrin and CD44 expression.

### CROSS-REFERENCE TO RELATED APPLICATION

The present application claims the benefit of U.S. Provisional Application Ser. No. 61/454,919, filed Mar. 21, 2011, which is hereby incorporated by reference herein in its entirety, including any figures, tables, or drawings.

### GOVERNMENT SUPPORT

This invention was made with Government support under Grant No. CA122065 awarded by the National Institutes of Health. The Government has certain rights in the invention.

### BACKGROUND OF THE INVENTION

Multiple myeloma is a disease characterized by the homing and uncontrolled growth of malignant plasma cells within the confines of the bone marrow (1, 2). Despite the recent advances in therapy, multiple myeloma remains an incurable disease. 14,000 new cases of multiple myeloma are diagnosed each year in the United States with a five year survival rate of 37% (3). Although standard therapy will typically cause an initial response, myeloma patients ultimately develop drug resistance and become unresponsive to a variety of anti-cancer agents, a phenomenon known as multidrug resistance (MDR). Clinical observations indicate that despite divergent genetic changes typical of myeloma, current therapy is not curative in any subset of patients. The bone marrow microenvironment presents a rich source of extracellular matrices, cytokines and growth factors produced by constituents residing in the bone marrow stroma, including mesenchymal stem cells, fibroblast and mature osteoblasts, suggesting that the bone marrow microenvironment may contribute to the resistant phenotype and the failure of standard chemotherapy. It has been reported that adhesion of myeloma and leukemia cells to components of the extracellular matrix is sufficient to cause drug resistance (4-13). Targeting interactions between the microenvironment and myeloma cells may be an attractive strategy for increasing the efficacy of standard therapy.

A D-amino acid containing peptide, referred to as HYD1, is known to block adhesion of prostate cells to extracellular matrices (14, 15). In addition to blocking adhesion of multiple myeloma cells to fibronectin, it was found that HYD1 induced caspase independent cell death in myeloma cell lines as a single agent in vitro and in vivo (16). Experimental evidence indicated that in prostate cancer cell lines that HYD1 interacts with a3 and a6 integrin (15).

### BRIEF SUMMARY OF THE INVENTION

It has been determined that the β1 integrin antagonist referred to as HYD1 induces necrotic cell death as a single agent in vitro and in-vivo using the SCID-Hu bone implant model. The inventors sought to delineate the determinants of sensitivity and resistance towards HYD1 induced cell death. To this end, the inventors developed a HYD1 isogenic resistant myeloma cell line by chronically exposing the parental cell line to increasing concentrations of HYD1.

In the studies described herein, the inventors show that the acquisition of resistance towards HYD1 correlates with reduced expression of the cleaved a4 integrin subunit. The reduction in a4 protein levels in the resistant cell line was found to be due to a post-transcriptional regulation of a4 integrin. Consistent with reduced VLA-4 (α4β1) expression, the resistant variant showed ablated functional binding to fibronectin, VCAM-1 and the bone marrow stroma cell line HS-5. The reduction in binding of the resistant cell line to HS-5 cells translated to increased sensitivity to melphalan and velcade induced cell death in the bone marrow stroma co-culture model of drug resistance. Importantly, the data herein show that HYD1 is more potent in relapsed myeloma specimens compared to newly diagnosed patients, a finding which correlated with a4 integrin expression. Collectively, these data indicate that the HYD1 peptide may represent a good candidate for pursing clinical trials in relapsed myeloma and in particular patients with high levels of a4 integrin and provide a further rationale for continued pre-clinical development of HYD1 and analogs of HYD1 for the treatment of multiple myeloma and potentially other tumors which home to the bone.

The subject invention concerns methods for treating a malignancy in a subject, comprising administering an effective amount of an agent that binds CD44, such as a HYD1 peptide, to the subject to treat the malignancy, wherein the malignancy has at least one of the following characteristics: the malignancy is a relapsing malignancy, the malignancy is one with elevated expression or activity of the a4 integrin subunit, and/or the malignancy expresses CD44. Expression levels of a4 integrin subunit and/or CD44 can be assessed by obtaining a sample of the malignancy from the patient and using methods known in the art for determining expression level of a biomarker at the transcript (mRNA) level, at the protein level, or both. Optionally, the method further comprises determining the presence of one or more of the aforementioned characteristics prior to administration of the peptide. In some embodiments, the malignancy is multiple myeloma or another hematologic malignancy. Optionally, the method further comprises administering an effective amount of an agent that increases the expression or activity of the a4 integrin subunit in cells of the malignancy (malignant cells). The agent may be administered before, simultaneously with, or after the CD44-binding agent (e.g., a HYD1 peptide).

Optionally, the diagnostic and treatment methods of the invention may further comprise assessing the presence or amount of a tumor-specific or tumor-related antigen of interest at the transcript (mRNA) level, at the protein level, or both, e.g., to confirm the cancer type. For example, CD138 may be used as a marker for myeloma, CD34 may be used as a marker for AML and CML, and cytokeratin 7 may be used as a marker for cancers of the breast, lung, and cervix. Other examples of tumor-specific or tumor-associated markers that may be used with the compositions and methods of the invention are provided in Table 1. It is anticipated that levels of CD44 and/or CD44 splice variants or alpha4 integrin on the cell surface will be used to identify patients likely to respond to agents (such as HYD1) that bind CD44 and induce necrotic cell death. Methods that may be utilized for detecting the levels of biomarkers of interest such as alpha4 integrin, CD44, and tumor-specific markers (e.g., CD138, CD34) include, but are not limited to, flow cytometry, immunohystochemistry (IHC), and LC-MS/MS multiple reaction monitoring.

The invention concerns methods for inhibiting the growth of a cancer cell in vitro or in vivo, comprising administering an effective amount of a HYD1 peptide to the cell in vitro or in vivo to inhibit cell growth, wherein the cancer cell has at least one of the following characteristics: wherein the malignancy has at least one of the following characteristics: the cancer cell is that of a relapsing cancer, the cancer cell is one with elevated expression or activity of the a4 integrin subunit, the cancer cell expresses CD44, and/or the cancer cell expresses CD138. Optionally, the method further comprises determining the presence of one or more of the aforementioned characteristics prior to administering the peptide. In some embodiments, the cancer cell is a multiple myeloma cell or a cell of another hematologic malignancy.

The invention also concerns methods for selecting agents that can enhance the cytotoxic response of a cancer cell to a HYD1 peptide based on a candidate agent's ability to increase the expression or activity of the a4 integrin subunit. In some embodiments, the selection method comprises selecting an agent that is predetermined to be effective in increasing the expression or activity of the a4 integrin subunit. In some embodiments, the selection method comprises determining whether a candidate agent increases the expression or activity of the a4 integrin subunit in a cancer cell in vitro or in vivo, and selecting the candidate agent for treatment if the candidate agent increases the expression or activity of the a4 integrin subunit the cancer cell in vitro or in vivo. In some embodiments, the cancer cell is a multiple myeloma cell or a cell of another hematologic malignancy.

The invention also concerns methods for determining whether a cancer will be sensitive or resistant to treatment with CD44 binding agent, such as HYD1 peptide (e.g., sensitive or resistant to HYD1-induced cell death), comprising assessing one or more of the following parameters in a cell sample of the cancer: expression or activity of the a4 integrin subunit, functional binding to fibronectin, functional binding to VCAM-1, CD44 expression, CD138 expression, and functional binding to HS-5 stromal cells; wherein one or more of reduced expression or activity of the a4 integrin subunit, lack of CD44 expression, lack of CD138 expression, reduced functional binding to fibronectin, reduced functional binding to VCAM-1, and reduced functional binding to HS-5 stromal cells) are indicative of resistance or lack of sensitivity; and wherein one or more of elevated expression or activity of the a4 integrin subunit, CD44 expression, CD138 expression, elevated functional binding to fibronectin, elevated functional binding to VCAM-1, and elevated functional binding to HS-5 stromal cells are indicative of sensitivity or lack of resistance. The cell sample may be, for example, a cancer cell line or primary cell sample. In some embodiments, the cancer cell sample is obtained from a subject having the cancer. In some embodiments, the cancer is multiple myeloma or another hematologic malignancy.

In some embodiments, the assessing step comprises determining the expression level or activity of the a4 integrin subunit, the expression level of CD44 expression, or both.

The invention also concerns methods for inhibiting the growth of a cancer cell in vitro or in vivo, comprising administering an effective amount of an agent that binds CD44, such as a HYD1 peptide, and an effective amount of an agent that increases the expression or activity of the a4 integrin subunit to the cell in vitro or in vivo to inhibit cell growth. The agent may be administered before, simultaneously with, or after the CD44-binding agent. In some embodiments, the cancer cell has at least one of the following characteristics: the cancer cell is that of a relapsing cancer, the cancer cell is one with elevated expression or activity of the a4 integrin subunit, the cancer cell expresses CD44, and/or the cancer cell expresses CD138. Optionally, the method further comprises determining the presence of one or more of the aforementioned characteristics prior to administering the peptide. In some embodiments, the cancer cell is a multiple myeloma cell or a cell of another hematologic malignancy.

The invention concerns methods for treating a malignancy in a subject, comprising administering to the subject an effective amount of a HYD1 peptide and an effective amount of an agent that increases the expression or activity of the a4 integrin subunit in cells of the malignancy (malignant cells). The agent may be administered before, simultaneously with, of after the HYD1 peptide. In some embodiments, the malignancy is one that exhibits reduced expression or activity of the a4 integrin subunit. In some embodiments, the malignancy has at least one of the following characteristics: the malignancy is a relapsing malignancy, the malignancy is one with elevated expression or activity of the a4 integrin subunit, the malignancy expresses CD44, and/or the malignancy expresses CD138. Optionally, the method further comprises determining the presence of one or more of the aforementioned characteristics prior to administering the peptide. In some embodiments, the malignancy is multiple myeloma or another hematologic malignancy.

The invention also concerns methods for treating a malignancy in a subject, comprising administering a HYD1 peptide and one or more anti-cancer agents to the subject. In some embodiments, the anti-cancer agent is one or more selected from suberoylanilide hydroxamic acid (SAHA) or other histone deacetylase inhibitor, arsenic trioxide, doxorubicin or other anthracycline DNA intercalating agent, and etoposide or other topoisomerase II inhibitor. In some embodiments, the anti-cancer agent is one or more listed in Table 2. The HYD1 peptide may be administered before, during, or after the one or more of the aforementioned agents. In some embodiments, the malignancy is multiple myeloma or another hematologic malignancy.

The invention also concerns a composition comprising a HYD1 peptide and an agent that increases the expression or activity of the a4 integrin subunit in a malignancy.

The invention also concerns a composition comprising a HYD1 peptide and an agent that decreases the expression or activity of the a4 integrin subunit in a malignancy. In some embodiments, the agent is an antibody (monoclonal or polyclonal) or antibody fragment against alpha 4 integrin, such as Natalizumab.

The invention also concerns a composition comprising a HYD1 peptide and one or more anti-cancer agents. In some embodiments, the anti-cancer agent is one or more selected from among suberoylanilide hydroxamic acid (SAHA) or other histone deacetylase inhibitor, arsenic trioxide, doxorubicin or other anthracycline DNA intercalating agent, and etoposide or other topoisomerase II inhibitor. In some embodiments, the anti-cancer agent is one or more listed in Table 2. The composition is useful for inhibiting the growth of cancer cells (for example, myeloma cells) in vitro or in vivo, when administered thereto. In those embodiments of the aforementioned methods of the invention in which another agent that is administered simultaneously with the HYD1 peptide, the agent and HYD1 peptide may be administered within the same formulation (e.g., in a composition of the invention) or in separate formulations.

Optionally, the methods of the invention further include the administration of additional agents, such as therapeutic or prophylactic agents, for example as anti-cancer agents (e.g., chemotherapeutic agents) or agents to treat or prevent infection (antibiotics, antimicrobials). Likewise, compositions of the invention may optionally include such additional agents.

The invention also concerns a cell line exhibiting resistance to HYD1 peptide-induced cell death, and isolated cells there from. In some embodiments, the cell line is a plasma cell line. In some embodiments, the cell line is a human cell line. In some embodiments, the cell line is a human plasma cell line. In one embodiment, the cell line is a variant H929 cell line. In some embodiments, the HYD1-resistant cell line exhibits reduced expression of the a4 integrin subunit.

The invention also concerns a method for producing a cell line with resistance to HYD1 peptide-induced cell death, comprising culturing a sensitive cell in the presence of increasing amounts of a HYD1 peptide for a period of time sufficient to produce a cell with resistance to HYD1 peptide-induced cell death. In some embodiments, the cell line is a plasma cell line. In some embodiments, the cell line is a human cell line. In some embodiments, the cell line is a human plasma cell line. In one embodiment, the cell line is a variant H929 cell line. In some embodiments, the resulting HYD1-resistant cell line exhibits reduced expression of the a4 integrin subunit.

Further details concerning the compositions and methods of the invention are provided in the Biological Assays and Assay Kits section herein.

As used herein, unless specified, “a HYD1 peptide” is inclusive of the d-amino acid peptide having the sequence: KIKMVISWKG (HYD1; SEQ ID NO:1), as well as other HYD1-related peptides (which includes d-amino acid containing peptides and non-d-amino acid containing peptides) disclosed, for example, in U.S. Pat. No. 7,632,814 (Hazelhurst et al., “HYD1 Peptides as Anti-Cancer Agents”), which is incorporated herein by reference in its entirety. In the Examples and Figures herein, reference to HYD1 refers to the all d-amino acid peptide having the sequence: KIKMVISWKG (HYD1; SEQ ID NO:1).

### BRIEF DESCRIPTION OF THE DRAWINGS

FIGS. 1A-1C. HYD1 is more potent in MM cells (H929) compared to normal hematopoietic cells. FIG. 1A: HYD1 treatment did not inhibit differentiation or colony formation of normal CD34 positive cells. FIG. 1B: Peripheral blood mononuclear cells (PBMC) cells were not sensitive to HYD1 induced cell death. FIG. 1C: H929 cells are more sensitive to HYD1 induced inhibition of colony growth compared to normal CD34 positive cells.

FIGS. 2A-2B. FIG. 2A: HYD1 has in vivo anti-tumor activity in the SCID-Hu in vivo model (p<0.05 repeated measures test). Tumor burden was measured by circulating kappa levels. FIG. 2B: HYD1 has anti-tumor activity in the murine 5TGM1 MM cell line.

FIGS. 3A-3D. Reducing a4 levels in MM cell lines (FIGS. 3A and 3C: 8226 and FIGS. 3B and 3D:H929) induced resistance towards HYD1 induced cell death. Experiment was performed in triplicates and three independent experiments were performed (p<0.05, Students t-test).

FIG. 4. Biotin-HYD1 interacts with CD44 in H929 cells. Thirty micrograms of membrane extract was incubated with either biotin or biotin-HYD1 bound NeutrAvidin beads. The first lane is 30 μg of membrane extract only. CD44 was detected by western blot analysis using a pan-CD44 antibody.

FIGS. 5A-5B. Biotin-HYD1 interacts with a4 integrin (FIG. 5A) and CD44 (FIG. 5B). Biotin-HYD1 or biotin was immobilized to NeutraAvidin beads prior to incubation with 150 μg of membrane extract. The blot was initially probed for a4 integrin and subsequently stripped and re-probed with CD44 antibody.

FIG. 6. Biotin-H Y D1 binds recombinant CD44 in a direct ELISA. A primary CD44 antibody and HRP conjugated secondary antibody and chemiluminescence detection was used to quantify rCD44 binding to immobilized biotin-HYD1.

FIGS. 7A-7B. HYD1 sensitivity in primary MM cells correlates with increased CD44 and a4 integrin expression. MM samples were initially immunosorted with CD138. Both CD138 positive and negative populations were screened for a4, 131 integrin and CD44 surface levels by FACS analysis (FIG. 7A). Cell death was measured 24 hours after exposure to HYD1 [100 ug/ml] using Topro-3 staining and FACS analysis (FIG. 7B).

FIG. 8. Table of combination index (CI) summary for combinations of HYD1 and SAHA, etoposide, arsenic, Vel, or doxorubicin in U226 cells.

FIGS. 9A-9B. NeutrAvidin/Biotin-HYD1 pull-down of a4 integrin (FIG. 9A) and CD44 (FIG. 9B) in H929 membrane lysate.

FIGS. 10A-10B. NeutrAvidin/Biotin-HYD1 pull-down comparison of H929 and H929-60 membrane lysate.

FIGS. 11A-11E. H929-60 cells are resistant to HYD1 induced cell death and demonstrated reduced binding of FAM-HYD1 to the cell surface. FIG. 11A: H929 and H929-60 cells were incubated with varying concentrations of HYD1 for 6 hours and HYD1 induced cell death was determined by Topro-3 staining and FACS analysis (ANOVA test p<0.05 n=9). FIG. 11B: Loss of mitochondrial membrane permeability was determined using DiOC6 staining following 2 hours of HYD1 treatment in H929 and H929-60 (* denotes p<0.05 n=9, Student's t-test). FIG. 11C: ATP levels were determined in the H929 and H929-60 cell lines following 6 hours of HYD1 (50 ug/ml) treatment. (* denotes p<0.05 and # denotes p>0.05, n=9, Student's t-test). FIGS. 11D and 11E: H929 cell (FIG. 11D) and H929-60 cells (FIG. 11E) were stained with Alexa Fluor 594 wheat germ agglutinin (WGA) and Hoechst 33342 for 30 minutes. 6.25 μg/ml FAM-HYD1 was added 10 minutes before images were taken. The experiment was repeated 3 independent times and shown is a representative experiment.

FIGS. 12A-12D. H929-60 cells have reduced a4 integrin protein levels and reduced adhesion to FN and VCAM-1. FIG. 12A: Surface expression of a4 and β1 integrin on H929 and H929-60 cells was determined by FACS analysis (* denotes p<0.05 n=9, Student's t-test).

FIGS. 12B-1 and 12B-2: Whole cell lystates of H929 cells (FIG. 12B-1) and H929-60 cells (FIG. 12B-2) were probed for a4 integrin, β1 integrin and beta actin by Western blot analysis. a4 integrin mRNA levels were determined using real time rt-PCR. a4 integrin mRNA expression levels were normalized by dividing by GAPDH levels (p>0.05 n=3). FIGS. 12C-12D: H929 and H929-60 cells were incubated with a4 blocking antibody or IgG control antibody for 30 minutes and subsequently adhered to (FIG. 12C) FN (40 μg/ml) or (FIG. 12D) VCAM-1 (10 μg/ml) coated plates for 2 hours. A representative of each experiment is shown (* denotes p<0.05 or # denotes p>0.05 Student's t-test).

FIGS. 13A1-13D. Reducing the expression of a4 and β1 integrins caused partial resistance to HYD1 induced cell death in H929 and 8226 cells. FIGS. 13A-1, 13A-2, 13C-1 and 13-C2: In H929 cells, a4 and β1 integrin expression was reduced via transient infection.

FIGS. 13A-1 and 13A-2: At 72 hours post-infection of shRNA, a4 and β1 integrin expression was determined by FACS analysis. 8226 cells were infected with a4 integrin shRNA, β1 integrin shRNA or control vector shRNA. FACS analysis was used to determine expression of a4 and β1 integrin. Shown is a representative histogram from one experiment.

FIGS. 13B and 13D: H929 and 8226 cells were treated with 50 μg/ml and 100 ug/ml of HYD1 respectively for 6 hours. After 6 hours, cell death was analyzed by Topro-3 staining and FACS analysis. (* denotes p<0.05 n=9, Student's t-test).

FIG. 14. Biotin-HYD1 interacts with a4 integrins and is attenuated in the resistant H929-60 cells. H929 and H929-60 membrane fractions were added to biotin or biotin-HYD1 bound NeutrAvidin beads for 18 hours as described in the methods and materials section. The experiment was repeated twice and shown is a representative experiment.

FIGS. 15A-15C. H929-60 cells display reduced binding to HS-5 stromal cells and are not resistant to velcade or melphalan in the stroma co-culture model. FIG. 15A: H929 and H929-60 pre-incubated with CMFDA dye were adhered to 10,000 HS-5 cells for 2 hours. After 2 hours unadherent cells were removed and the fluorescence intensity was measured on a fluorescent plate reader. FIGS. 15B and 15C: H929 and H929-60 cells were adhered to HS-5 GFP cells for 24 hours. After 24 hours samples were treated with 15 μM melphalan or 16 nM velcade for 24 hours. Cell death was analyzed using a FACScalibur with the GFP expressing HS-5 cells being excluded from the analysis. In FIGS. 15A-15C, a representative of three independent experiments was shown. (*=p<0.05, n=9 or#=p>0.05, n=9 Students t-test).

FIGS. 16A-16D. HYD1 induced cell death in CD138+ patient samples correlates with a4 integrin expression. FIG. 16A: CD138+ and CD138- cells were treated with 100 ug/ml HYD1 for 24 hours. After 24 hours cell death was measured by Topro-3 staining and FACS analysis. (p<0.05, Student's t-test). FIGS. 16B: a4 integrin expression was compared to HYD1 induced cell death using a Pearson's correlation coefficient. The CD138+ population demonstrated a significant correlation between a4 integrin expression and HYD1 induced cell death (p<0.05). FIGS. 16C and 16D: Specimens were separated into two groups depending on the clinical diagnosis of either; newly diagnosed or specimens obtained from patients that were considered relapsed. In FIG. 16C, CD138+ cells were treated with 100 ug/ml HYD1 for 24 hours. After 24 hours cell death was measured by Topro-3 staining and FACS analysis (Student's t-test p<0.05). In FIG. 16D, CD138+ cells were used to analyze for a4 integrin expression by FACS analysis. (Student's t-test p<0.05).

FIG. 17. HYD1 induced levels of ROS are decreased in H929-60 cells compared to H929 cells. ROS production was measured using the dye 5-(and-6)-carboxy-2′,Tdichlorodihydro-fluorescein diacetate. H929 and H929-60 cells (4×10⁵ cells/ml) were treated for various time points (0, 30, 60 and 120 minutes) with 50 μg/ml HYD1 or VC(H₂O). Dye fluorescence intensity was analyzed using Wallac VICTOR21420 multilabel counter (EG&G Wallac, Turku, Finland) (excitation: 485 nm, emission: 535 nm). A representative of three independent experiments is shown.

FIGS. 18A, 18B-1, 18B-2, and 18B-3. H929-60 cells have decreased FAM-HYD1 binding compared to H929 cells. FIG. 18A: H929 and H929-60 cells (4×10⁵ cells/ml) were incubated with FAM-HYD1 (6.25, 12.5 μg/ml) on ice for 15 minutes. FACS analysis was used to determine the geometric mean of fluorescence of FAM-HYD1 bound to H929 and H929-60 cells. A representative of three independent experiments is shown. A student's t-test was performed to determine significance (* denotes p<0.05). FIGS. 18B-1, 18B-2, and 18B-3: When the dose of FAM-HYD1 was increased to 12.5 μg/ml FAM-HYD1 binding to plasma membrane was detected in the resistant cell line as determine by confocal microscopy.

FIG. 19. H929-60 cells are not resistant to standard myeloma therapies. H929 and H929-60 cells were treated with increasing concentrations of melphalan or Velcade for 24 hours. After 24 hours, cells were stained with annexin V FITC/P1 for 45 minutes and acquired on FACScan. IC50 values were determined by linear regressions. Comparisons between H929 and H929-60 were not deemed to be significant using a students t-test (p>0.05, n=9). Shown in the table of FIG. 19 is the mean and standard deviations of three independent experiments.

FIGS. 20A-B. Representative sensorgrams from surface plasmon resonance (SPR) assay of HYD1 binding kinetics to the human CD44 ectodomain. The colored lines represent the raw data while the superimposed black lines represent the 1:1 (Langmuir) binding model fit (shown in FIG. 20A). Peak RU increases with the concentration rCD44 (0.6 nM to 400 nM). The experiment was repeated 4 times, and shown in the table in FIG. 20B is the association, dissociation, and KD values for each experiment

### BRIEF DESCRIPTION OF THE SEQUENCES

SEQ ID NO: 1 is the amino acid sequence KIKMVISWKG (HYD1).

SEQ ID NO: 2 is the amino acid sequence AIAMVISWAG (HYD8).

SEQ ID NO: 3 is the amino acid sequence AIKMVISWKG (HYD6).

SEQ ID NO: 4 is the amino acid sequence AIKMVISWKG (HUD2).

SEQ ID NO: 5 is the amino acid sequence AKMVISW.

SEQ ID NO: 6 is the amino acid sequence AKMVISWKG.

SEQ ID NO: 7 is the amino acid sequence IAMVISW.

SEQ ID NO: 8 is the amino acid sequence IAMVISWKG.

SEQ ID NO: 9 is the amino acid sequence IKAVISW.

SEQ ID NO: 10 is the amino acid sequence IKAVISWKG.

SEQ ID NO: 11 is the amino acid sequence IKMAISW.

SEQ ID NO: 12 is the amino acid sequence IKMAISWKG.

SEQ ID NO: 13 is the amino acid sequence IKMVASW.

SEQ ID NO: 14 is the amino acid sequence IKMVASWKG.

SEQ ID NO: 15 is the amino acid sequence IKMVIAW.

SEQ ID NO: 16 is the amino acid sequence IKMVIAWKG.

SEQ ID NO: 17 is the amino acid sequence IKMVISA.

SEQ ID NO: 18 is the amino acid sequence IKMVISAKG.

SEQ ID NO: 19 is the amino acid sequence IKMVISW.

SEQ ID NO: 20 is the amino acid sequence IKMVISWAG.

SEQ ID NO: 21 is the amino acid sequence KMVISWKA.

SEQ ID NO: 22 is the amino acid sequence IKMVISWKG (HYD18; (-K)HYD1).

SEQ ID NO: 23 is the amino acid sequence ISWKG.

SEQ ID NO: 24 is the amino acid sequence KAKMVISWKG.

SEQ ID NO: 25 is the amino acid sequence KIAMVISWAG (HYD7).

SEQ ID NO: 26 is the amino acid sequence KIAMVISWKG.

SEQ ID NO: 27 is the amino acid sequence KIKAVISWKG.

SEQ ID NO: 28 is the amino acid sequence KIKMAISWKG.

SEQ ID NO: 29 is the amino acid sequence KIKMV.

SEQ ID NO: 30 is the amino acid sequence KIKMVASWKG.

SEQ ID NO: 31 is the amino acid sequence KIKMVI (HYD16).

SEQ ID NO: 32 is the amino acid sequence KIKMVIAWKG.

SEQ ID NO: 33 is the amino acid sequence KIKMVIS (HYD15).

SEQ ID NO: 34 is the amino acid sequence KIKMVISAKG.

SEQ ID NO: 35 is the amino acid sequence KIKMVISW (HYD14).

SEQ ID NO: 36 is the amino acid sequence KIKMVISWAG.

SEQ ID NO: 37 is the amino acid sequence KIKMVISWK (HYD17; HYD1(-G)).

SEQ ID NO: 38 is the amino acid sequence KIKMVISWKA.

SEQ ID NO: 39 is the amino acid sequence KMVISWKG (HYD9).

SEQ ID NO: 40 is the amino acid sequence LSWKG (HYD12).

SEQ ID NO: 41 is the amino acid sequence MVISWKG (HYD10).

SEQ ID NO: 42 is the amino acid sequence SWKG (HYD13).

SEQ ID NO: 43 is the amino acid sequence VISWKG (HYD11).

SEQ ID NO: 44 is the amino acid sequence WIKSMKIVKG.

SEQ ID NO: 45 is the amino acid sequence KMVIXW.

SEQ ID NO: 46 is the amino acid sequence IKMVISWXX.

SEQ ID NO: 47 is the amino acid sequence KMVISWXX.

SEQ ID NO:48 is the amino acid sequence XKMVISWXX

### DETAILED DISCLOSURE OF THE INVENTION

Multiple myeloma (MM) is the second most common hematopoietic malignancy. The majority of MM patients will initially respond to standard chemotherapeutic treatments, but the failure to eliminate minimal residual disease (MRD) and the emergence of drug resistance results in a median survival time of 5 years. MRD is typically found in the bone marrow compartment, suggesting that this particular microenvironment provides the survival signals that contribute to failure to eliminate MRD and the emergence of refractory disease. Clinical outcomes strongly support the need for novel target identification and therapeutic agent development to treat MM long-term and the inventors propose that targeting MM-stromal cell interactions of the tumor microenvironment are important to the goal of eliminating MRD. The inventors have shown that adhesion of hematopoietic tumor cells to the extracellular matrix fibronectin (FN) is sufficient to induce resistance to chemotherapeutic agents with diverse structures and mechanisms of action. The inventors referred to this phenotype as cell adhesion mediated drug resistance or CAM-DR. Moreover, it has been found that the bone marrow microenvironment provides a sanctuary for diverse tumors including multiple myeloma (MM), acute myeloid leukemia (AML), chronic lymphocytic leukemia (CLL), lymphoma, chronic myelogenous leukemia (CML), lung, and breast cancer. In summary, the data indicate that disruption of tumor-stromal cell adhesion is a viable strategy for increasing the efficacy of standard therapy in many types of solid and hematopoietic tumors. Myeloma is a disease that homes to the bone marrow, and it is well established that the bone marrow microenvironment contributes to progression and the intrinsic drug resistance associated with the disease.

Using combinatorial peptide libraries and a functional binding assay, several peptides that inhibited adhesion of DU145 prostate cancer cells to fibronectin, laminin and collagen IV have previously been identified. The lead candidate, isolated from the functional screens, was the D-amino acid peptide referred to as HYD1 (KIKMVISWKG; SEQ ID NO:1). HYD1 was shown to inhibit integrin dependent binding of epithelial prostate carcinoma cells to extracellular matrixes (i.e., laminin 5, laminin 332, collagen and fibronectin). In contrast, a scrambled derivative of HYD1 referred to as HYD1S (WIKSMKIVKG; (SEQ ID NO:44) was shown not to inhibit cell adhesion to extracellular matrixes. The inhibition of cell adhesion was not attributed to cell death or cell cycle arrest but rather to dissociation of adhesion and the resulting downstream signaling events. The inventors recently reported that HYD1 blocks a4β1 mediated adhesion of MM cells to the extracellular matrix fibronectin. Many members of the integrin family including avβ3, a5β1, a8β1 and a4β1 recognize an Arg-Gly-Asp (RGD) motif. RGD containing peptides have been shown to competitively block integrin ligand interactions. Furthermore, treatment of leukemic cells with RGD containing peptides was shown to induce apoptotic cell death independent of the presence of ligand. HYD1 lacks the RGD motif, but the inventors compared and contrasted HYD1 activities with that of known RGD ligand activities. In a recently published manuscript, the inventors show that HYD1 treatment induces cell death in MM cells in a matrix independent fashion as a single agent in vitro and retained activity in vivo (Nair R R, Emmons M F, Cress A E, et al. HYD1-induced increase in reactive oxygen species leads to autophagy and necrotic cell death in multiple myeloma cells. Mol Cancer Ther 2009 August; 8(8):2441-51). However, unlike RGD containing peptides, HYD1 treatment does not cause apoptotic cell death; it does not trigger the activation of caspases, the release of apoptosis-inducing factor (AIF), and Endonuclease G (Endo G) from the mitochondria. Furthermore, HYD1 treatment does not induce double stranded DNA breaks indicative of activation of endonucleases. Rather, HYD1 causes necrosis as shown by: (a) a decrease in mitochondrial membrane potential (Δψₘ); (b) a loss of total cellular ATP, and; (c) an increase in reactive oxygen species (ROS) production. HYD1 does initiate autophagy in MM cells; however, autophagy is an adaptive response rather than the cause of cell death. The inventors also show that N-acetyl-L-cysteine (NAC), a thiol containing free radical scavenger, partially protects MM cells against HYD1-induced death. Importantly, HYD1 is inactive against normal CD34 positive and mononuclear cells. Finally, as predicted, HYD1 treatment increases the efficacy of melphalan treatment in the bone marrow stroma co-culture model of drug resistance.

CD44 is a family of cell surface receptors that mediate cell-cell and cell-matrix adhesion. One gene encodes the CD44 family, but alternative splicing leads to multiple variants which is thought to be in part responsible for the apparent diverse functions attributed to this single pass membrane spanning cell surface protein. CD44 facilitates cell adhesion and metastasis, augments growth factor signaling and expression of CD44 was shown to protect against hypoxia induced lung injury. CD44 can be shed from the cell membrane and soluble CD44 is associated with advanced disease in chronic lymphocytic leukemia. Hyaluronic acid (HA) is the most predominant ligand for CD44. HA is an abundant polysaccharide found in extracellular matrices. The structure of CD44 consists of a constant region spanning the first five exons which defines the HA binding domain and is found in all splice variants. Exon 7 through 15 (corresponding to v2-v10), is the variable region of the molecule. This region is localized on the external domain and adds to the stem region of CD44. The highly conserved cytoplasmic region has part of exon 18 combined with exon 19 and 20. HYD1 sensitive cell lines H929 and U226 express the standard foam of CD44 as well as cd44v3 and CD44v9. CD44 variant expression is associated with markers of cancer stem cells and is a poor prognostic indicator for many cancer types including myeloma. CD44v associates with a4β1 integrin in chronic lymphocytic leukemia (CLL) and positively regulates adhesion of myeloma cells to stroma and extracellular matrices. Finally, it is known that CD44 associates with VLA-4 integrin in T-cells and CLL. Based on the inventors' recent data presented herein using biotin-HYD1 to pull down membrane complexes, the inventors propose that HYD1 interacts directly with CD44 and requires a functional CD44/a4β1 complex to mediate cell death. In this application, strong data is provided that supports the finding that biotin-HYD1 interacts with CD44/a4β1 integrin containing complex in HYD1 sensitive MM cell lines.

HYD1 was previously shown to inhibit integrin dependent binding of epithelial prostate carcinoma cells to extracellular matrixes (i.e., laminin 5, laminin 332, collagen and fibronectin). In these studies, the inhibition of cell adhesion was not attributed to cell death or cell cycle arrest but due to the dissociation of adhesion and signaling events. Similarly, the inventors recently reported that HYD1 blocks a4β1 mediated adhesion of MM cells to fibronectin (Nair et al. 2009). The inventors further investigated whether HYD1 treatment was sufficient to induce cell death in MM cells. The results indicated that HYD1, but not the scrambled peptide, induced cell death in U226, MM1.S and 8226 cells. Additionally, the co-culture bone marrow stroma model of drug resistance was not found to confer resistance to HYD1 treatment. Finally HYD1 treatment potentiated melphalan induced cell death in both suspension and co-culture models of cell growth. Together, these data indicate that HYD1 (a) has anti-tumor activity as a single agent (b) is equally potent when MM cells are grown as a suspension culture or in a co-culture system and (c) reverses drug resistance associated with the bone marrow stroma co-culture model system.

In order to delineate the molecular pathway of HYD1 induced caspase independent cell death, the inventors developed an acquired isogenic HYD1 resistant H929 myeloma cell line referred to as H929-60 cells. The data herein show that the acquisition of resistance towards HYD1 does not result in a phenotype that is cross-resistant to other agents used to treat myeloma, including bortezomib and melphalan. The data also show that resistance correlates with reduced a4 integrin expression and ablated functional binding to fibronectin and VCAM-1 as well as to the bone marrow stroma cell line HS-5. The attenuation of a4 integrin is mediated by a post-transcriptional regulation in the resistant cell line. Moreover, acquisition of resistance towards HYD1 occurs at a cost in overall fitness, as the resistant variant demonstrates reduced binding to extracellular matrixes and is not resistant to melphalan or velcade induced cell death in the bone marrow co-culture model system. Finally, the data show that specimens obtained from relapsed/refractory patient were significantly more sensitive to HYD1 induced cell death compared to specimens obtained from newly diagnosed patients. The data show that HYD1 is an attractive agent for treating multiple myeloma patients and may be an important strategy for the treatment of relapsed disease.

To further characterize the mechanism of HYD1 induced cell death, an isogenic drug resistant variant was developed. This variant, called H929-60, was developed by chronically exposing parental H929 cells to increasing doses of HYD1 over time until a drug resistant phenotype was observed. The resistant cell lines demonstrated reduced binding of FAM-conjugated HYD1 to the cell surface compared to the parental cell line. The inventors previously reported that HYD1 blocks a4B 1 mediated adhesion to fibronectin and thus the inventors asked whether resistance towards HYD1 induced cell death correlated with reduced a4β1 integrin expression. The resistant variant showed deceased levels of a4 integrin protein expression despite no change in RNA levels, indicating a post-transcriptional regulation of a4 expression. These changes were consistent with decreased binding to fibronectin, VCAM-1 and HS-5 stromal cells. Moreover, the inventors demonstrated that biotin conjugated HYD1 is able to pull down an a4 containing complex and the amount of a4 bound to biotin-HYD1 was reduced in the drug resistant variant. Reducing the expression of a4 and β1 integrins on the cell surface in H929 cells was sufficient to confer partial resistance to HYD1 induced cell death. Because the H929 resistant variant showed reduced adhesion to stroma cells, the inventors asked whether acquisition of resistance towards HYD1 resulted in reduced levels of drug resistance in the HS-5 co-culture model of drug resistance. Using the HS-5 co-culture model, the inventors show that the drug resistant variant H929-60 cells treated with either melphalan or velcade- were drug sensitive in the HS-5 stromal co-culture model of drug resistance. Thus, the drug resistant variant failed to demonstrate a cell adhesion mediate drug resistance (CAM-DR), indicating that as cells acquire resistance to HYD1, they lose resistance initiated by the tumor microenvironment. Finally, the inventors show that HYD1 was significantly more potent in relapsed/refractory patient specimens compared to newly diagnosed patient specimens. Additionally, the inventors show that a4 integrin expression positively correlates with HYD1 sensitivity. Together, these data indicate that HYD1 is an attractive agent for the treatment of relapsed/refractory multiple myeloma with high levels of a4 integrin expression.

The subject invention concerns methods for treating a malignancy in a subject, comprising administering an effective amount of an agent that binds CD44, such as a HYD1 peptide, to the subject to treat the malignancy, wherein the malignancy has at least one of the following characteristics: the malignancy is a relapsing malignancy, the malignancy is one with elevated expression or activity of the a4 integrin subunit, and/or the malignancy expresses CD44. Expression levels of a4 integrin subunit and/or CD44 can be assessed by obtaining a sample of the malignancy from the patient and using methods known in the art for determining expression level of a biomarker at the transcript (mRNA) level, at the protein level, or both. Optionally, the method further comprises determining the presence of one or more of the aforementioned characteristics prior to administration of the CD44-binding agent. In some embodiments, the malignancy is multiple myeloma or another hematologic malignancy. Optionally, the method further comprises administering an effective amount of an agent that increases the expression or activity of the a4 integrin subunit in cells of the malignancy (malignant cells). The agent may be administered before, simultaneously with, or after the CD44-binding agent (e.g., a HYD1 peptide).

Optionally, the diagnostic and treatment methods of the invention may further comprise assessing the presence or amount of a tumor-specific or tumor-associated antigen of interest at the transcript (mRNA) level, at the protein level, or both, e.g., to confirm the cancer type. For example, CD138 may be used as a marker for myeloma, CD34 may be used as a marker for AML and CML, and cytokeratin 7 may be used as a marker for cancers of the breast, lung, and cervix. Other examples of tumor-specific or tumor-associated markers that may be used with the compositions and methods of the invention are provided in Table 1. It is anticipated that levels of CD44 and/or CD44 splice variants or Alpha 4 integrin on the cell surface will be used to identify patients likely to respond to agents (such as HYD1) that bind CD44 and induce necrotic cell death. Methods that may be utilized for detecting the levels of biomarkers of interest such as alpha4 integrin, CD44, and tumor-specific markers (e.g., CD138, CD34) include, but are not limited to, flow cytometry, immunohystochemistry (IHC), and LC-MS/MS multiple reaction monitoring. Further details concerning the methods of the invention are provided in the Biological Assays and Assay Kits section herein.

TABLE 1
Exemplified Tumor Markers

| Tumor marker | Associated tumor types |
|---|---|
| Alpha fetoprotein (AFP) | germ cell tumor, hepatocellular carcinoma |
| CA15-3 | breast cancer |
| CA19-9 | Mainly pancreatic cancer, but also colorectal |
| | cancer and other types of gastrointestinal |
| | cancer. |
| CA-125 | Mainly ovarian cancer, but may also be |
| | elevated in for example endometrial cancer, |
| | fallopian tube cancer, lung cancer, breast |
| | cancer and gastrointestinal cancer. May also |
| | increase in endometriosis. |
| Calretinin | mesothelioma, sex cord-gonadal stromal |
| | tumour, adrenocortical carcinoma, synovial |
| | sarcoma |
| Carcinoembryonic antigen | gastrointestinal cancer, cervix cancer, lung |
| | cancer, ovarian cancer, breast cancer, urinary |
| | tract cancer |
| CD34 | hemangiopericytoma/solitary fibrous tumor, |
| | pleomorphic lipoma, gastrointestinal stromal |
| | tumor, dermatofibrosarcoma protuberans |
| CD99 | Ewing sarcoma, primitive neuroectodermal |
| | tumor, hemangiopericytoma/solitary fibrous |
| | tumor, synovial sarcoma, lymphoma, |
| | leukemia, sex cord-gonadal stromal tumour |
| CD117 | gastrointestinal stromal tumor, mastocytosis, |
| | seminoma |
| Chromogranin | neuroendocrine tumor |
| Cytokeratin (various types) | Many types of carcinoma, some types of |
| | sarcoma |
| Desmin | smooth muscle sarcoma, skeletal muscle |
| | sarcoma, endometrial stromal sarcoma |
| Epithelial membrane protein (EMA) | many types of carcinoma, meningioma, some |
| | types of sarcoma |
| Factor VIII, CD31 FL1 | vascular sarcoma |
| Glial fibrillary acidic protein (GFAP) | glioma (astrocytoma, ependymoma) |
| Gross cystic disease fluid protein | breast cancer, ovarian cancer, salivary gland |
| (GCDFP-15) | cancer |
| HMB-45 | melanoma, PEComa (for example |
| | angiomyolipoma), clear cell carcinoma, |
| | adrenocortical carcinoma |
| Human chorionic gonadotropin (hCG) | gestational trophoblastic disease, germ cell |
| | tumor, carcinoma |
| immunoglobulin | lymphoma, leukemia |
| inhibin | sex cord-gonadal stromal tumor, adrenocortical |
| | carcinoma, hemangioblastoma |
| keratin (various types) | carcinoma, some types of sarcoma |
| PTPRC (CD45) | lymphoma, leukemia, histiocytic tumor |
| lymphocyte marker (various types) | lymphoma, leukemia |
| MART-1 (Melan-A) | melanoma, steroid-producing tumors |
| | (adrenocortical carcinoma, gonadal tumor) |
| Myo D1 | rhabdomyosarcoma, small, round, blue cell |
| | tumor |
| muscle-specific actin (MSA) | myosarcoma (leiomyosarcoma, |
| | rhabdomyosarcoma) |
| neurofilament | neuroendocrine tumor, small-cell carcinoma of |
| | the lung |
| neuron-specific enolase (NSE) | neuroendocrine tumor, small-cell carcinoma of |
| | the lung, breast cancer |
| placental alkaline phosphatase (PLAP) | seminoma, dysgerminoma, embryonal |
| | carcinoma |
| prostate-specific antigen | prostate |
| S100 protein | melanoma, sarcoma (neurosarcoma, lipoma, |
| | chondrosarcoma), astrocytoma, gastrointestinal |
| | stromal tumor, salivary gland cancer, some |
| | types of adenocarcinoma, histiocytic tumor |
| | (dendritic cell, macrophage) |
| smooth muscle actin (SMA) | gastrointestinal stromal tumor, |
| | leiomyosarcoma, PEComa |
| synaptophysin | neuroendocrine tumor |
| thyroglobulin | thyroid cancer (but not in medullary thyroid |
| | cancer) |
| thyroid transcription factor-1 | all types of thyroid cancer, lung cancer |
| Tumor M2-PK | colorectal cancer, breast cancer, renal cell |
| | carcinoma, lung cancer, pancreatic cancer, |
| | esophageal cancer, stomach cancer, cervical |
| | cancer, ovarian cancer |
| vimentin | sarcoma, renal cell carcinoma, endometrial |
| | cancer, lung carcinoma, lymphoma, leukemia, |
| | Melanoma |

The invention concerns methods for inhibiting the growth of a cancer cell in vitro or in vivo, comprising administering an effective amount of a HYD1 peptide to the cell in vitro or in vivo to inhibit cell growth, wherein the cancer cell has at least one of the following characteristics: wherein the malignancy has at least one of the following characteristics: the cancer cell is that of a relapsing cancer, the cancer cell is one with elevated expression or activity of the a4 integrin subunit, the cancer cell expresses CD44, and/or the cancer cell expresses CD138. Optionally, the method further comprises determining the presence of one or more of the aforementioned characteristics prior to administering the peptide. In some embodiments, the cancer cell is a multiple myeloma cell or a cell of another hematologic malignancy.

The invention also concerns methods for selecting agents that can enhance the cytotoxic response of a cancer cell to a HYD1 peptide based on a candidate agent's ability to increase the expression or activity of the a4 integrin subunit. In some embodiments, the selection method comprises selecting an agent that is predetermined to be effective in increasing the expression or activity of the a4 integrin subunit. In some embodiments, the selection method comprises determining whether a candidate agent increases the expression or activity of the a4 integrin subunit in a cancer cell in vitro or in vivo, and selecting the candidate agent for treatment if the candidate agent increases the expression or activity of the a4 integrin subunit the cancer cell in vitro or in vivo. In some embodiments, the cancer cell is a multiple myeloma cell or a cell of another hematologic malignancy.

The invention also concerns methods for determining whether a cancer will be sensitive or resistant to treatment with CD44 binding agent, such as HYD1 peptide (e.g., sensitive or resistant to HYD1-induced cell death), comprising assessing one or more of the following parameters in a cell sample of the cancer: expression or activity of the a4 integrin subunit, functional binding to fibronectin, functional binding to VCAM-1, CD44 expression, CD138 expression, and functional binding to HS-5 stromal cells; wherein one or more of reduced expression or activity of the a4 integrin subunit, lack of CD44 expression, lack of CD138 expression, reduced functional binding to fibronectin, reduced functional binding to VCAM-1, and reduced functional binding to HS-5 stromal cells) are indicative of resistance or lack of sensitivity; and wherein one or more of elevated expression or activity of the a4 integrin subunit, CD44 expression, CD138 expression, elevated functional binding to fibronectin, elevated functional binding to VCAM-1, and elevated functional binding to HS-5 stromal cells are indicative of sensitivity or lack of resistance. Further details concerning the methods of the invention are provided in the Biological Assays and Assay Kits section herein.

The cell sample may be, for example, a cancer cell line or primary cell sample. In some embodiments, the cancer cell sample is obtained from a subject having the cancer. In some embodiments, the cancer is multiple myeloma or another hematologic malignancy.

In some embodiments, the assessing step comprises determining the expression level or activity of the a4 integrin subunit, the expression level of CD44 expression, or both.

The invention also concerns methods for inhibiting the growth of a cancer cell in vitro or in vivo, comprising administering an effective amount of an agent that binds CD44, such as a HYD1 peptide, and an effective amount of an agent that increases the expression or activity of the a4 integrin subunit to the cell in vitro or in vivo to inhibit cell growth. The agent may be administered before, simultaneously with, or after the CD44-binding agent. In some embodiments, the cancer cell has at least one of the following characteristics: the cancer cell is that of a relapsing cancer, the cancer cell is one with elevated expression or activity of the a4 integrin subunit, the cancer cell expresses CD44, and/or the cancer cell expresses CD138. Optionally, the method further comprises determining the presence of one or more of the aforementioned characteristics prior to administering the peptide. In some embodiments, the cancer cell is a multiple myeloma cell or a cell of another hematologic malignancy.

The invention concerns methods for treating a malignancy in a subject, comprising administering to the subject an effective amount of a CD44 binding agent, such as a HYD1 peptide, and an effective amount of an agent that increases the expression or activity of the a4 integrin subunit in cells of the malignancy (malignant cells). The agent may be administered before, simultaneously with, of after the CD44-binding agent (e.g., before, during, or after administration of a HYD1 peptide). In some embodiments, the malignancy is one that exhibits reduced expression or activity of the a4 integrin subunit. In some embodiments, the malignancy has at least one of the following characteristics: the malignancy is a relapsing malignancy, the malignancy is one with elevated expression or activity of the a4 integrin subunit, the malignancy expresses CD44, and/or the malignancy expresses CD138. Optionally, the method further comprises determining the presence of one or more of the aforementioned characteristics prior to administering the peptide. In some embodiments, the malignancy is multiple myeloma or another hematologic malignancy.

The invention also concerns methods for treating a malignancy in a subject, comprising administering a HYD1 peptide and one or more anti-cancer agents to the subject. In some embodiments, the anti-cancer agent is one or more selected from suberoylanilide hydroxamic acid (SAHA) or other histone deacetylase inhibitor, arsenic trioxide, doxorubicin or other anthracycline DNA intercalating agent, and etoposide or other topoisomerase II inhibitor. In some embodiments, the anti-cancer agent is one or more listed in Table 3. The HYD1 peptide may be administered before, during, or after the one or more of the aforementioned agents. In some embodiments, the malignancy is multiple myeloma or another hematologic malignancy.

The invention also concerns a composition comprising a HYD1 peptide and an agent that increases the expression or activity of the a4 integrin subunit in a malignancy.

The invention also concerns a composition comprising a HYD1 peptide and an agent that decreases the expression or activity of the a4 integrin subunit in a malignancy. In some embodiments, the agent is an antibody (monoclonal or polyclonal) or antibody fragment against alpha 4 integrin, such as Natalizumab.

The invention also concerns a composition comprising an agent that binds CD44, such as a HYD1 peptide, and one or more anti-cancer agents. In some embodiments, the anti-cancer agent is one or more selected from among suberoylanilide hydroxamic acid (SAHA) or other histone deacetylase inhibitor, arsenic trioxide, doxorubicin or other anthracycline DNA intercalating agent, and etoposide or other topoisomerase II inhibitor. In some embodiments, the anti-cancer agent is one or more listed in Table 3. The composition is useful for inhibiting the growth of cancer cells (for example, myeloma cells) in vitro or in vivo, when administered thereto.

In those embodiments of the aforementioned methods of the invention in which another agent that is administered simultaneously with the CD44-binding agent (e.g., with the HYD1 peptide), the additional agent and CD44-binding agent may be administered within the same formulation (e.g., in a composition of the invention) or in separate formulations.

Optionally, the methods of the invention further include the administration of additional agents, such as therapeutic or prophylactic agents, for example as anti-cancer agents (e.g., chemotherapeutic agents) or agents to treat or prevent infection (antibiotics, antimicrobials). Likewise, compositions of the invention may optionally include such additional agents.

The invention also concerns a cell line exhibiting resistance to HYD1 peptide-induced cell death, and isolated cells there from. In some embodiments, the cell line is a plasma cell line. In some embodiments, the cell line is a human cell line. In some embodiments, the cell line is a human plasma cell line. In one embodiment, the cell line is a variant H929 cell line. In some embodiments, the HYD1-resistant cell line exhibits reduced expression of the a4 integrin subunit.

The invention also concerns a method for producing a cell line with resistance to HYD1 peptide-induced cell death, comprising culturing a sensitive cell in the presence of increasing amounts of a HYD1 peptide for a period of time sufficient to produce a cell with resistance to HYD1 peptide-induced cell death. In some embodiments, the cell line is a plasma cell line. In some embodiments, the cell line is a human cell line. In some embodiments, the cell line is a human plasma cell line. In one embodiment, the cell line is a variant H929 cell line. In some embodiments, the resulting HYD1-resistant cell line exhibits reduced expression of the a4 integrin subunit.

The invention also concerns an array that may be used to assess expression of biomarkers of interest within a sample (e.g., alpha4 integrin, CD44, and tumor antigens) in accordance with the treatment and diagnostic methods of the invention. In some embodiments, the array comprises a substrate and two or more capture probes disposed thereon, wherein the two or more capture probes comprise or consist of:

(a) antibodies, or antibody fragments, that specifically bind alpha4 integrin and CD44; or

(b) oligonucleotides that are partially or fully complementary to, and bind to, nucleic acid sequences encoding alpha4 integrin and CD44.

The substrate may be any suitable support for the capture probes that may be contacted with a sample. The substrate may be any solid or semi-solid carrier for supporting the capture probes, such as a particle (e.g., magnetic or latex particle), a microtiter multi-well plate, a bead, a slide, a filter, a chip, a membrane, a cuvette, or a reaction vessel.

In some embodiments, the substrate comprises a particle, a microtiter multi-well plate, a bead, a membrane, a cuvette, or a reaction vessel. In some embodiments, the array further comprises one or more capture probes comprising or consisting of:

(c) antibodies, or antibody fragments, that specifically bind a tumor-specific or tumor-associated antigen; or

(d) oligonucleotides that are partially or fully complementary to, and bind to, nucleic acid sequences encoding a tumor-specific or tumor-associated antigen. In some embodiments, the tumor-specific or tumor-associated antigen is selected from among CD138, CD34, cytokeratin 7, or a tumor marker listed in Table 1. In some embodiments, the array comprises less than 50 capture probes (i.e., capture probes for less than 50 different target molecules). In some embodiments, the array comprises less than 100 capture probes (i.e., capture probes for less than 100 different target molecules). In some embodiments, the array comprises less than 500 capture probes (i.e., capture probes for less than 500 different target molecules).

### Biological Assays and Assay Kits

In certain embodiments, the samples are assayed for assessing one or more biomarkers of the invention. The biomarker and biomarkers useful according to the present invention (e.g., a4 integrin subunit, CD44, CD138) can be determined by methods including, but not limited to, enzyme-linked immunosorbant assays (ELISA), Western blot, immunological assays, microarrays, radioimmunoassays (RIAs), lateral flow assays, immunochromatographic strip assays, automated flow assays, immunoprecipitation assays, reversible flow chromatographic binding assays, agglutination assays, Southern blots, immunofluorescence, flow cytometry, immunocytochemistry, nucleic acid hybridization techniques, nucleic acid reverse transcription methods, nucleic acid amplification, polymerase chain reaction (PCR), DNA arrays, protein arrays, mass spectrometry, and any combination thereof. In addition, immune cell populations and profiles are routinely examined using flow cytometry analysis.

The level and/or the presence of the biomarkers can be determined either at the nucleic acid (such as mRNA) or protein level. In some embodiments, the expression of a biomarker is detected on a protein level using, for example, antibodies that are directed against specific biomarker proteins. These antibodies can be used in various methods such as Western blot, ELISA, immunoprecipitation, immunocytochemistry, flow cytometry, and cell sorting (FACS). Reduction in biomarker gene expression can be detected at the mRNA level by techniques including, but not limited to, real-time RT-PCR, microarray analysis, and Northern blotting. Preferably, all expression data is compared with levels of a “house keeping” gene to normalize for variable amounts of RNA in different samples.

In one embodiment of the method of the invention, the assessing step comprises: (a) contacting the sample with a binding agent that binds biomarker protein to form a complex; (b) detecting the complex; and (c) correlating the detected complex to the amount of biomarker protein in the sample. In a specific embodiment, the detecting of (b) further comprises linking or incorporating a label onto the agent, or using ELISA-based immunoenzymatic detection.

In another embodiment of the method of the invention, the assessing step comprises: (a) contacting the sample with a binding agent that binds biomarker nucleic acid (e.g., mRNA) to form a complex; (b) detecting the complex; and (c) correlating the detected complex to the amount of biomarker nucleic acid in the sample. In a specific embodiment, the detecting of (b) further comprises linking or incorporating a label onto the agent, or using ELISA-based immunoenzymatic detection.

The terms “detecting” or “detect” include assaying or otherwise establishing the presence or absence of the target biomarker (e.g., a4 integrin subunit, CD44, CD138), subunits thereof, or combinations of agent bound targets, and the like. The term encompasses quantitative, semi-quantitative, and qualitative detection methodologies. In embodiments of the invention involves detection of biomarker protein (as opposed to nucleic acid molecules encoding biomarker protein). In one embodiment the detection method is an ELISA-based method. Preferably, in the various embodiments of the invention, the detection method provides an output (i.e., readout or signal) with information concerning the presence, absence, or amount of the biomarker in a sample. For example, the output may be qualitative (e.g., “positive” or “negative”), or quantitative (e.g., a concentration such as nanograms per milliliter).

In one embodiment, the assessing step comprises the following steps:

(a) incubating a biological sample with a first antibody specific for the biomarker protein (e.g., a4 integrin subunit, CD44, tumor antigen such as CD138, CD34, cytokeratin 7) which is directly or indirectly labeled with a detectable substance, and a second antibody specific for the first antibody;

(b) separating the first antibody from the second antibody to provide a first antibody phase and a second antibody phase;

(c) detecting the detectable substance in the first or second antibody phase thereby quantitating the biomarker in the sample; and

(d) comparing the quantitated biomarker level with a standard.

As is known in the art, polypeptides or proteins in test samples are commonly detected with immunoassay devices and methods. Alternatively, or additionally, aptamers can be selected and used for binding of even greater specificity, as is well known in the art.

These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Specific immunological binding of the antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is pre-bound to the antibody, and a change in signal is measured as the unlabelled antigen displaces the bound, labeled antigen from the receptor.

Lateral flow assays can be conducted according to the teachings of U.S. Pat. No. 5,712,170 and the references cited therein. U.S. Pat. No. 5,712,170 and the references cited therein are hereby incorporated by reference in their entireties. Displacement assays and flow immunosensors useful for carrying out displacement assays are known. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration.

The use of immobilized antibodies specific for the biomarkers is also contemplated by the present invention and is well known by one of ordinary skill in the art. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

The analysis of a plurality of biomarkers may be carried out separately or simultaneously with one test sample. Several biomarkers may be combined into one test for efficient processing of a multiple of samples. In addition, one skilled in the art would recognize the value of testing multiple samples (for example, at successive time points) from the same individual. Such testing of serial samples will allow the identification of changes in biomarker levels over time.

The analysis of biomarkers can be carried out in a variety of physical formats as well. For example, the use of microtiter plates or automation can be used to facilitate the processing of large numbers of test samples. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and capillary devices.

In one embodiment of the invention, a sandwich enzyme-linked immunosorbent assay (ELISA) can be developed using monoclonal antibodies specific for the biomarkers of the invention. This assay can be used to detect the presence or levels of (e.g., a4 integrin subunit, CD44, tumor antigen such as CD138, CD34, cytokeratin 7) in cell samples.

In one embodiment, the assessing step in the assays (methods) of the invention can involve contacting, combining, or mixing the sample and the solid support, such as a reaction vessel, microvessel, tube, microtube, well, multi-well plate, or other solid support.

The methods of the invention can be carried out on a solid support. The solid supports used may be those which are conventional for the purpose of assaying an analyte in a sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. The biomarkers can be accessed in a sample in vivo or in vitro (ex vivo).

Samples and/or binding agents may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or “physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or 1536-well plates (or trays).

In another embodiment, the present invention provides a kit for the analysis of biomarkers. Such a kit preferably comprises devices and reagents for the analysis of at least one test sample and instructions for performing the assay. The kit may contain aptamers specific for a target biomarker. Optionally the kits may contain one or more means for using information obtained from immunoassays performed for a biomarker panel. Biomarker antibodies or antigens may be incorporated into immunoassay kits depending upon which biomarker autoantibodies or antigens are being measured. A first container may include a composition comprising an antigen or antibody preparation. Both antibody and antigen preparations should preferably be provided in a suitable titrated form, with antigen concentrations and/or antibody titers given for easy reference in quantitative applications.

The kits may also include an immunodetection reagent or label for the detection of specific immunoreaction between the provided antigen and/or antibody, as the case may be, and the sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label. Immunodetection reagents and processes suitable for application in connection with the novel methods of the present invention are generally well known in the art.

The reagents may also include ancillary agents such as buffering agents and protein stabilizing agents, e.g., polysaccharides and the like. The kit may further include where necessary agents for reducing background interference in a test, agents for increasing signal, software and algorithms for combining and interpolating biomarker values to produce a prediction of clinical outcome of interest, apparatus for conducting a test, calibration curves and charts, standardization curves and charts, and the like.

The measurement of the concentration of the biomarker in the sample may employ any suitable the biomarker antibody or aptamer to detect the protein. Such aptamers or antibodies may be presently extant in the art or presently used commercially, or may be developed by techniques now common in the field of immunology.

As used herein, the term “antibody” refers to an intact immunoglobulin having two light and two heavy chains or any antibody fragments thereof sufficient to bind a target of interest. Thus a single isolated antibody or antibody fragment may be a polyclonal antibody, a high affinity polyclonal antibody, a monoclonal antibody, a synthetic antibody, a recombinant antibody, a chimeric antibody, a humanized antibody, or a human antibody.

The term “antibody fragment,” as used herein, refers to less than an intact antibody structure, including, without limitation, an isolated single antibody chain, an Fv construct, a Fab construct, a light chain variable or complementarity determining region (CDR) sequence, etc. A recombinant molecule bearing the binding portion of an antibody, e.g., carrying one or more variable chain CDR sequences that bind the biomarker, may also be used in the detection assay of this invention.

Similarly the particular assay format used to measure the biomarker in a sample may be selected from among a wide range of immunoassays, such as enzyme-linked immunoassays, such as those described in the examples below, sandwich immunoassays, homogeneous assays, or other assay conventional assay formats. One of skill in the art may readily select from any number of conventional immunoassay formats to perform this invention.

Other reagents for the detection of protein in samples, such as peptide mimetics, synthetic chemical compounds capable of detecting the biomarker may be used in other assay formats for the quantitative detection in samples, such as Western blots, flow cytometry, etc.

As indicated above, kits of the invention include reagents for use in the methods described herein, in one or more containers. The kits may include primers, specific internal controls, and/or probes, buffers, and/or excipients, separately or in combination. Each reagent can be supplied in a solid form or liquid buffer that is suitable for inventory storage. Kits may also include means for obtaining a sample from a host organism or an environmental sample.

Kits of the invention can be provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like. Preferably, the solid matrix is a structure having a surface that can be derivatized to anchor an oligonucleotide probe, primer, molecular beacon, specific internal control, etc. Preferably, the solid matrix is a planar material such as the side of a microtiter well or the side of a dipstick. In certain embodiments, the kit includes a microtiter tray with two or more wells and with reagents including primers, probes, specific internal controls, and/or molecular beacons in the wells.

Kits of the invention may optionally include a set of instructions in printed or electronic (e.g., magnetic or optical disk) form, relating information regarding the components of the kits and/or how to make various determinations (e.g., biomarker levels, comparison to control standards, etc.). The kit may also be commercialized as part of a larger package that includes instrumentation for measuring other biochemical components.

In some embodiments, the antibodies can be labeled with pairs of FRET dyes, bioluminescence resonance energy transfer (BRET) protein, fluorescent dye-quencher dye combinations, and beta gal complementation assays protein fragments. The antibodies may participate in FRET, BRET, and fluorescence quenching or beta-gal complementation to generate fluorescence, colorimetric or enhanced chemiluminescence (ECL) signals, for example.

As used herein, the term “in vitro” has its art recognized meaning, e.g., involving purified reagents or extracts, e.g., cell extracts. The term “in vivo” also has its art recognized meaning, e.g., involving living cells in an organism, e.g., immortalized cells, primary cells, and/or cell lines, in an organism.

Agents that are capable of detecting the biomarker in the samples of subjects are those that interact or bind with the biomarker polypeptide or the nucleic acid molecule encoding the biomarker. Examples of such agents (also referred to herein as binding agents) include, but are not limited to, antibodies or fragments thereof that bind to the biomarker, binding partners, and nucleic acid molecules that hybridize to the nucleic acid molecules encoding the biomarker polypeptides. Preferably, the binding agent is labeled with a detectable substance (e.g., a detectable moiety). The binding agent may itself function as a label.

In addition, the binding of the HYD1 peptide to the biomarkers of the invention (e.g., a4 integrin subunit, CD44, CD138) can be determined by standard binding assays. Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

One approach is to use solubilized, unpurified or solubilized purified polypeptide or peptides, for example extracted from transformed eukaryotic or prokaryotic host cells. This allows for a “molecular” binding assay with the advantages of increased specificity, the ability to automate, and high drug test throughput.

Ligand-binding assays provide a direct method for ascertaining receptor pharmacology and are adaptable to a high throughput format. The purified ligand for a receptor may be radiolabeled to high specific activity (50-2000 Ci/mmol) for binding studies. A determination is then made that the process of radiolabeling does not diminish the activity of the ligand towards its receptor. Assay conditions for buffers, ions, pH and other modulators such as nucleotides are optimized to establish a workable signal to noise ratio for both membrane and whole cell receptor sources. For these assays, specific receptor binding is defined as total associated radioactivity minus the radioactivity measured in the presence of an excess of unlabeled competing ligand. Where possible, more than one competing ligand is used to define residual nonspecific binding.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

“Specific binding” or “specificity” refers to the ability of an antibody or other agent to detectably bind an epitope presented on an antigen, such as a HYD1, a4 integrin subunit, CD44, or a cancer-specific biomarker such as CD138, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

“Selectivity” refers to the preferential binding of a protein to a particular region, target, or peptide as opposed to one or more other biological molecules, structures, cells, tissues, etc. For example, selectivity can be determined by competitive ELISA or Biacore assays. The difference in affinity/avidity that marks selectivity can be any detectable preference (e.g., a ratio of more than 1:1.1, or more than about 1:5, if detectable.

As used herein, the term “a4 integrin” is inclusive of the cleaved form of a4 integrin and the a4β1 complex, and may be read to include either or both.

“CD44” is a multi-structural glycoprotein involved in many physiological and pathological functions, including cell-cell and cell-matrix adhesion, support of cell migration, presentation of growth factors, chemokines or enzymes to corresponding cell surface receptors or relevant substrates, as well as transmission of signals from the membrane to the cytoskeleton or nucleus (Naor, D., et al. Adv. Cancer Res. 71, 241-319, (1997); Lesley, J., et al. Adv. Immunol. 54, 271-335, (1993)]. This glycoprotein is known to bind to multiple ligands (e.g. fibrinogen, fibronectin, alanine, collagen), a principal one being hyaluronic acid (HA).

As used herein, the term “multiple myeloma” is inclusive of relapsing or recurring multiple myeloma and newly diagnosed multiple myeloma.

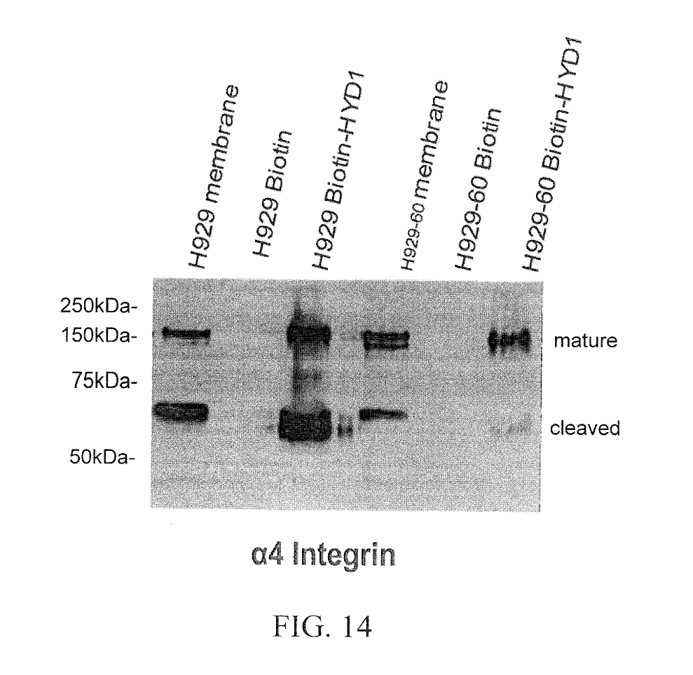

In some embodiments of the invention, the HYD1 peptide is a peptide comprising or consisting of the amino acid sequence KIKMVISWKG (HYD1; SEQ ID NO:1) or a HYD1-related peptide from among AIAMVISWAG (SEQ ID NO:2; HYD8); AIKMVISWKG (SEQ ID NO:3; HYDE); AIKMVISWKG (SEQ ID NO:4; HYD2); AKMVISW (SEQ ID NO:5); AKMVISWKG (SEQ ID NO:6); IAMVISW (SEQ ID NO:7); IAMVISWKG (SEQ ID NO:8); IKAVISW (SEQ ID NO:9); IKAVISWKG (SEQ ID NO:10); IKMAISW (SEQ ID NO:11); IKMAISWKG (SEQ ID NO:12); IKMVASW (SEQ ID NO:13); IKMVASWKG (SEQ ID NO:14); IKMVIAW (SEQ ID NO:15); IKMVIAWKG (SEQ ID NO:16); IKMVISA (SEQ ID NO:17); IKMVISAKG (SEQ ID NO:18); IKMVISW (SEQ ID NO:19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWAG (SEQ ID NO:25; HYD7); KIAMVISWKG (SEQ ID NO:26); KIKMVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIAWKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYD1(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYD10); SWKG (SEQ ID NO:42; HYD13); VISWKG (SEQ ID NO:43; HYD11); WIKSMKIVKG (SEQ ID NO:44); KMVIXW (SEQ ID NO:45); IKMVISWXX (SEQ ID NO:46); KMVISWXX (SEQ ID NO:47); and XKMVISWXX (SEQ ID NO:48); wherein X is any amino acid (traditional or non-traditional amino acid). In another embodiment, the peptide consists of the amino acid sequence. In another embodiment, the peptide consists essentially of the amino acid sequence. In another embodiment, the peptide is one listed in the figures in U.S. Pat. No. 7,632,814. In one embodiment, the peptide is one of the variants listed in FIG. 14A-14C, 15A-15C, or 16A-1-16C-2 of U.S. Pat. No. 7,632,814 that is substituted with an alanine at one position, and wherein another residue is substituted in place of the alanine. Preferably, the residue is a conservative substitution. In some embodiments, the peptide is a linear peptide.

The peptide can comprise at least one D-amino acid. For example, depending upon the number of amino acids in the peptide, the peptide may include one, two, three, four, five, six, seven, eight, nine, or ten or more D-amino acids. Preferably, each amino acid of the peptide is a D-amino acid.

The methods of the present invention can be advantageously combined with at least one additional treatment method, including but not limited to, chemotherapy, radiation therapy, or any other therapy known to those of skill in the art for the treatment and management of a cancer.

While peptides of the invention can be administered to cells in vitro and in vivo as isolated agents, it is preferred to administer these peptides as part of a pharmaceutical composition. The subject invention thus further provides compositions comprising a peptide of the invention in association with at least one pharmaceutically acceptable carrier. The pharmaceutical composition can be adapted for various routes of administration, such as enteral, parenteral, intravenous, intramuscular, topical, subcutaneous, intratumoral, and so forth. Administration can be continuous or at distinct intervals, as can be determined by a person of ordinary skill in the art.

The peptides of the invention can be formulated according to known methods for preparing pharmaceutically useful compositions. Formulations are described in a number of sources which are well known and readily available to those skilled in the art. For example, Remington's Pharmaceutical Science (Martin, E.W., 1995, Easton Pa., Mack Publishing Company, 19^{th} ed.) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

Pharmaceutically acceptable salts of compounds may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

As used herein, the term “analogs” refers to compounds which are substantially the same as another compound but which may have been modified by, for example, adding side groups, oxidation or reduction of the parent structure. Analogs of the HYD1 peptide, and other peptides disclosed herein, can be readily prepared using commonly known standard reactions. These standard reactions include, but are not limited to, hydrogenation, alkylation, acetylation, and acidification reactions. Chemical modifications can be accomplished by those skilled in the art by protecting all functional groups present in the molecule and deprotecting them after carrying out the desired reactions using standard procedures known in the scientific literature (Greene, T. W. and Wuts, P.G.M. “Protective Groups in Organic Synthesis” John Wiley & Sons, Inc. New York. 3rd Ed. pg. 819, 1999; Honda, T. et al. Bioorg. Med. Chem. Lett., 1997, 7:1623-1628; Honda, T. et al. Bioorg. Med. Chem. Lett., 1998, 8:2711-2714; Konoike, T. et al. J. Org. Chem., 1997, 62:960-966; Honda, T. et al. J. Med. Chem., 2000, 43:4233-4246; each of which are hereby incorporated herein by reference in their entirety). Analogs, fragments, and variants of the HYD1 peptide exhibiting the desired biological activity (such as induction of apoptosis, cytotoxicity, cytostaticity, induction of cell cycle arrest, etc.) can be identified or confirmed using cellular assays or other in vitro or in vivo assays.

CD44 binding agents such as peptides may be locally administered at one or more anatomical sites, such as sites of unwanted cell growth (such as a tumor site, e.g., injected intratumorally or topically applied to the tumor), optionally in combination with a pharmaceutically acceptable carrier such as an inert diluent. Peptides of the invention may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the peptides may be incorporated into sustained-release preparations and devices.

The active agent (CD44-binding agents such as HYD1 peptides) may also be administered intravenously or intraperitoneally by infusion or injection. Solutions of the active agent can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds of the invention which are adapted for the extemporaneous preparation of sterile injectable or infusible solutions or dispersions, optionally encapsulated in liposomes. The ultimate dosage form. should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating the peptides of the invention in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the agents may be applied in pure-form, i.e., when they are liquids. However, it will generally be desirable to administer them topically to the skin as compositions, in combination with a dermatologically acceptable carrier, which may be a solid or a liquid.

The agents of the subject invention can be applied topically to a subject's skin to reduce the size (and may include complete removal) of malignant or benign growths. The peptides of the invention can be applied directly to the growth. Preferably, the peptide is applied to the growth in a formulation such as an ointment, cream, lotion, solution, tincture, or the like. Drug delivery systems for delivery of pharmacological substances to dermal lesions can also be used, such as that described in U.S. Pat. No. 5,167,649 (Zook).

Useful solid carriers include finely divided solids such as talc, clay, microcrystalline cellulose, silica, alumina and the like. Useful liquid carriers include water, alcohols or glycols or water-alcohol/glycol blends, in which the peptide can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the peptides to the skin are disclosed in Jacquet et al. (U.S. Pat. No. 4,608,392), Geria (U.S. Pat. No. 4,992,478), Smith et al. (U.S. Pat. No. 4,559,157) and Woltzman (U.S. Pat. No. 4,820,508).

Useful dosages of the pharmaceutical compositions of the present invention can be determined by comparing their in vitro activity, and in vivo activity in animal models. Methods for the extrapolation of effective dosages in mice, and other animals, to humans are known in the art; for example, see U.S. Pat. No. 4,938,949.

Accordingly, the present invention includes a pharmaceutical composition comprising a peptide of the invention (or encoding polynucleotide operably linked with a promoter for expression) in combination with a pharmaceutically acceptable carrier. Pharmaceutical compositions adapted for oral, topical or parenteral administration, comprising an amount of a compound of the invention constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Depending upon the disorder or disease condition to be treated, a suitable dose(s) may be that amount that will reduce proliferation or growth of the target cell(s), or induce cell death. In the context of cancer, a suitable dose(s) is that which will result in a concentration of the active agent (the compound of the invention) in cancer tissue, such as a malignant tumor, which is known to achieve the desired response. The preferred dosage is the amount which results in maximum inhibition of cancer cell growth, without unmanageable side effects. Administration of a peptide (or encoding polynucleotide) of the invention can be continuous or at distinct intervals, as can be determined by a person of ordinary skill in the art.

To provide for the administration of such dosages for the desired therapeutic treatment, in some embodiments, pharmaceutical compositions of the invention can comprise between about 0.1% and 45%, and especially, 1 and 15%, by weight of the total of one or more of the compounds of the invention based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Mammalian species which benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cells of such human and non-human species.

Patients in need of treatment using the methods of the present invention can be identified using standard techniques known to those in the medical or veterinary professions, as appropriate.

As used herein, the terms “cancer” and “malignancy” are used inclusively. As used herein, the terms “cancer” and “malignancy” refer to or describe the physiological condition in mammals that is typically characterized by unregulated cell growth. The cancer may be drug-resistant or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer. In some embodiments, the cancer is multiple myeloma or another hematologic malignancy. In some embodiments, the cancer is a hematopoietic cancer.

In some embodiments, the cancer or malignancy is one that expresses alpha4 integrin subunit, CD44, or both. In some embodiments, the methods of the invention further comprises obtaining a sample of the cancer cells directly from the subject or from a third party (e.g., a healthcare provider) and determining (assessing) whether the cells express CD44 and/or alpha4 integrin prior to administration of a peptide of the invention. Optionally, the methods may further comprise administering the peptide if the cancer sample expresses CD44, alpha4 integrin, or both. Optionally, the method further comprises assessing whether the cell expresses a marker that is specific for the cancer (e.g., CD138 for multiple myeloma).

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas.

Examples of cancer in the context of the invention are listed in Table 2 below.

TABLE 2
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult (Primary) |
| Acute Myeloid Leukemia, Adult | Hepatocellular (Liver) Cancer, Childhood |
| Acute Myeloid Leukemia, Childhood | (Primary) |
| Adrenocortical Carcinoma | Hodgkin's Lymphoma, Adult |
| Adrenocortical Carcinoma, Childhood | Hodgkin's Lymphoma, Childhood |
| AIDS-Related Cancers | Hodgkin's Lymphoma During Pregnancy |
| AIDS-Related Lymphoma | Hypopharyngeal Cancer |
| Anal Cancer | Hypothalamic and Visual Pathway Glioma, |
| Astrocytoma, Childhood Cerebellar | Childhood |
| Astrocytoma, Childhood Cerebral | Intraocular Melanoma |
| Basal Cell Carcinoma | Islet Cell Carcinoma (Endocrine Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, Osteosarcoma/Malignant | Laryngeal Cancer |
| Fibrous Histiocytoma | Laryngeal Cancer, Childhood |
| Brain Stem Glioma, Childhood | Leukemia, Acute Lymphoblastic, Adult |
| Brain Tumor, Adult | Leukemia, Acute Lymphoblastic, Childhood |
| Brain Tumor, Brain Stem Glioma, | Leukemia, Acute Myeloid, Adult |
| Childhood | Leukemia, Acute Myeloid, Childhood |
| Brain Tumor, Cerebellar Astrocytoma, | Leukemia, Chronic Lymphocytic |
| Childhood | Leukemia, Chronic Myelogenous |
| Brain Tumor, Cerebral | Leukemia, Hairy Cell |
| Astrocytoma/Malignant Glioma, | Lip and Oral Cavity Cancer |
| Childhood | Liver Cancer, Adult (Primary) |
| Brain Tumor, Ependymoma, Childhood | Liver Cancer, Childhood (Primary) |
| Brain Tumor, Medulloblastoma, | Lung Cancer, Non-Small Cell |
| Childhood | Lung Cancer, Small Cell |
| Brain Tumor, Supratentorial Primitive | Lymphoma, AIDS-Related |
| Neuroectodermal Tumors, Childhood | Lymphoma, Burkitt's |
| Brain Tumor, Visual Pathway and | Lymphoma, Cutaneous T-Cell, see Mycosis |
| Hypothalamic Glioma, Childhood | Fungoides and Sézary Syndrome |
| Brain Tumor, Childhood | Lymphoma, Hodgkin's, Adult |
| Breast Cancer | Lymphoma, Hodgkin's, Childhood |
| Breast Cancer, Childhood | Lymphoma, Hodgkin's During Pregnancy |
| Breast Cancer, Male | Lymphoma, Non-Hodgkin's, Adult |
| Bronchial Adenomas/Carcinoids, | Lymphoma, Non-Hodgkin's, Childhood |
| Childhood | Lymphoma, Non-Hodgkin's During Pregnancy |
| Burkitt's Lymphoma | Lymphoma, Primary Central Nervous System |
| Carcinoid Tumor, Childhood | Macroglobulinemia, Waldenström's |
| Carcinoid Tumor, Gastrointestinal | Malignant Fibrous Histiocytoma of |
| Carcinoma of Unknown Primary | Bone/Osteosarcoma |
| Central Nervous System Lymphoma, | Medulloblastoma, Childhood |
| Primary | Melanoma |
| Cerebellar Astrocytoma, Childhood | Melanoma, Intraocular (Eye) |
| Cerebral Astrocytoma/Malignant Glioma, | Merkel Cell Carcinoma |
| Childhood | Mesothelioma, Adult Malignant |
| Cervical Cancer | Mesothelioma, Childhood |
| Childhood Cancers | Metastatic Squamous Neck Cancer with Occult |
| Chronic Lymphocytic Leukemia | Primary |
| Chronic Myelogenous Leukemia | Multiple Endocrine Neoplasia Syndrome, |
| Chronic Myeloproliferative Disorders | Childhood |
| Colon Cancer | Multiple Myeloma/Plasma Cell Neoplasm |
| Colorectal Cancer, Childhood | Mycosis Fungoides |
| Cutaneous T-Cell Lymphoma, see | Myelodysplastic Syndromes |
| Mycosis Fungoides and Sézary | Myelodysplastic/Myeloproliferative Diseases |
| Syndrome | Myelogenous Leukemia, Chronic |
| Endometrial Cancer | Myeloid Leukemia, Adult Acute |
| Ependymoma, Childhood | Myeloid Leukemia, Childhood Acute |
| Esophageal Cancer | Myeloma, Multiple |
| Esophageal Cancer, Childhood | Myeloproliferative Disorders, Chronic |
| Ewing's Family of Tumors | Nasal Cavity and Paranasal Sinus Cancer |
| Extracranial Germ Cell Tumor, | Nasopharyngeal Cancer |
| Childhood | Nasopharyngeal Cancer, Childhood |
| Extragonadal Germ Cell Tumor | Neuroblastoma |
| Extrahepatic Bile Duct Cancer | Non-Hodgkin's Lymphoma, Adult |
| Eye Cancer, Intraocular Melanoma | Non-Hodgkin's Lymphoma, Childhood |
| Eye Cancer, Retinoblastoma | Non-Hodgkin's Lymphoma During Pregnancy |
| Gallbladder Cancer | Non-Small Cell Lung Cancer |
| Gastric (Stomach) Cancer | Oral Cancer, Childhood |
| Gastric (Stomach) Cancer, Childhood | Oral Cavity Cancer, Lip and |
| Gastrointestinal Carcinoid Tumor | Oropharyngeal Cancer |
| Germ Cell Tumor, Extracranial, | Osteosarcoma/Malignant Fibrous Histiocytoma |
| Childhood | of Bone |
| Germ Cell Tumor, Extragonadal | Ovarian Cancer, Childhood |
| Germ Cell Tumor, Ovarian | Ovarian Epithelial Cancer |
| Gestational Trophoblastic Tumor | Ovarian Germ Cell Tumor |
| Glioma, Adult | Ovarian Low Malignant Potential Tumor |
| Glioma, Childhood Brain Stem | Pancreatic Cancer |
| Glioma, Childhood Cerebral | Pancreatic Cancer, Childhood |
| Astrocytoma | Pancreatic Cancer, Islet Cell |
| Glioma, Childhood Visual Pathway and | Paranasal Sinus and Nasal Cavity Cancer |
| Hypothalamic | Parathyroid Cancer |
| Skin Cancer (Melanoma) | Penile Cancer |
| Skin Carcinoma, Merkel Cell | Pheochromocytoma |
| Small Cell Lung Cancer | Pineoblastoma and Supratentorial Primitive |
| Small Intestine Cancer | Neuroectodermal Tumors, Childhood |
| Soft Tissue Sarcoma, Adult | Pituitary Tumor |
| Soft Tissue Sarcoma, Childhood | Plasma Cell Neoplasm/Multiple Myeloma |
| Squamous Cell Carcinoma, see Skin | Pleuropulmonary Blastoma |
| Cancer (non-Melanoma) | Pregnancy and Breast Cancer |
| Squamous Neck Cancer with Occult | Pregnancy and Hodgkin's Lymphoma |
| Primary, Metastatic | Pregnancy and Non-Hodgkin's Lymphoma |
| Stomach (Gastric) Cancer | Primary Central Nervous System Lymphoma |
| Stomach (Gastric) Cancer, Childhood | Prostate Cancer |
| Supratentorial Primitive | Rectal Cancer |
| Neuroectodermal Tumors, Childhood | Renal Cell (Kidney) Cancer |
| T-Cell Lymphoma, Cutaneous, see | Renal Cell (Kidney) Cancer, Childhood |
| Mycosis Fungoides and Sézary | Renal Pelvis and Ureter, Transitional Cell |
| Syndrome | Cancer |
| Testicular Cancer | Retinoblastoma |
| Thymoma, Childhood | Rhabdomyosarcoma, Childhood |
| Thymoma and Thymic Carcinoma | Salivary Gland Cancer |
| Thyroid Cancer | Salivary Gland Cancer, Childhood |
| Thyroid Cancer, Childhood | Sarcoma, Ewing's Family of Tumors |
| Transitional Cell Cancer of the Renal | Sarcoma, Kaposi's |
| Pelvis and Ureter | Sarcoma, Soft Tissue, Adult |
| Trophoblastic Tumor, Gestational | Sarcoma, Soft Tissue, Childhood |
| Unknown Primary Site, Carcinoma of, | Sarcoma, Uterine |
| Adult | Sezary Syndrome |
| Unknown Primary Site, Cancer of, | Skin Cancer (non-Melanoma) |
| Childhood | Skin Cancer, Childhood |
| Unusual Cancers of Childhood | |
| Ureter and Renal Pelvis, Transitional Cell | |
| Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

As used herein, the terms “administering” or “administer” is defined as the introduction of a substance into cells in vitro or into the body of an individual in vivo and includes oral, nasal, ocular, rectal, vaginal and parenteral routes. Compositions (e.g., peptides or polynucleotides encoding the peptides) may be administered individually or in combination with other agents via any route of administration, including but not limited to subcutaneous (SQ), intramuscular (IM), intravenous (IV), intraperitoneal (IP), intradermal (ID), via the nasal, ocular or oral mucosa (IN), or orally. For example, the peptides or nucleic acids can be administered by direct injection into a tumor, or systemically, into the circulatory system, to kill circulating tumor cells (CTC).

In the context of the instant invention, the terms “oligopeptide”, “polypeptide”, “peptide” and “protein” can be used interchangeably; however, it should be understood that the invention does not relate to the peptides in natural form, that is to say that they are not in their natural environment but that the polypeptides may have been isolated or obtained by purification from natural sources or obtained from host cells prepared by genetic manipulation (e.g., the peptides, or fragments thereof, are recombinantly produced by host cells, or by chemical synthesis). Peptides according to the instant invention may also contain non-natural amino acids, as will be described below. The terms “oligopeptide”, “polypeptide”, “peptide” and “protein” are also used, in the instant specification, to designate a series of residues of any length, typically L-amino acids, connected one to the other, typically by peptide bonds between the α-amino and carboxyl groups of adjacent amino acids. Linker elements can be joined to the polypeptides of the subject invention through peptide bonds or via chemical bonds (e.g., heterobifunctional chemical linker elements) as set forth below. Additionally, the terms “amino acid(s)” and “residue(s)” can be used interchangeably.

“Nucleotide sequence”, “polynucleotide” or “nucleic acid” can be used interchangeably and are understood to mean, according to the present invention, either a double-stranded DNA, a single-stranded DNA or products of transcription of the said DNAs (e.g., RNA molecules). It should also be understood that the present invention does not relate to genomic polynucleotide sequences in their natural environment or natural state.

As used herein, the terms “treat” or “treatment” refer to both therapeutic treatment and prophylactic or preventative measures, wherein the object is to prevent or slow down (lessen) an undesired physiological change or disorder, such as the development or spread of cancer or other proliferation disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. For example, treatment with a peptide of the invention may include reduction of undesirable cell proliferation, and/or induction of apoptosis and cytotoxicity. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the patient may be identified (e.g., diagnosed) as one suffering from the disease or condition (e.g., cancer) prior to administration of the peptide of the invention.

As used herein, the term “(therapeutically) effective amount” refers to an amount of the peptide of the invention or other agent (e.g., a drug) effective to treat a disease or disorder in a mammal. In the case of cancer or other proliferation disorder, the therapeutically effective amount of the agent may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; reduce β1 integrin signaling in the target cells, and/or relieve, to some extent, one or more of the symptoms associated with the cancer. To the extent the administered peptide prevents growth of and/or kills existing cancer cells, it may be cytostatic and/or cytotoxic. For cancer therapy, efficacy can, for example, be measured by assessing the time to disease progression (TTP) and/or determining the response rate (RR).

As used herein, the term “growth inhibitory amount” of the peptide of the invention refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

The terms “cell” and “cells” are used interchangeably herein and are intended to include either a single cell or a plurality of cells, in vitro or in vivo, unless otherwise specified.

As used herein, the term “anti-cancer agent” refers to a substance or treatment (e.g., radiation therapy) that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY). The anti-cancer agent administered before, during, after administration of the peptide. In one embodiment, the anti-cancer agent administered before, during, after administration of the peptide or encoding polynucleotide of the invention is melphalen. Anti-cancer agents include but are not limited to the chemotherapeutic agents listed Table 3.

As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, Tenn.), and anti-androgens such as flutamide, nilutamide, bicalutamide, leuprolide, and goserelin, etc. Examples of chemotherapeutic agents that may be used in conjunction with the compounds of the invention are listed in Table 3. In some embodiments, the chemotherapeutic agent is one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN).

TABLE 3
Examples of Chemotherapeutic Agents

| | |
|---|---|
| 13-cis-Retinoic Acid | Mylocel |
| 2-Amino-6- | Letrozole |
| Mercaptopurine | Neosar |
| 2-CdA | Neulasta |
| 2-Chlorodeoxyadenosine | Neumega |
| 5-fluorouracil | Neupogen |
| 5-FU | Nilandron |
| 6-TG | Nilutamide |
| 6-Thioguanine | Nitrogen Mustard |
| 6-Mercaptopurine | Novaldex |
| 6-MP | Novantrone |
| Accutane | Octreotide |
| Actinomycin-D | Octreotide acetate |
| Adriamycin | Oncospar |
| Adrucil | Oncovin |
| Agrylin | Ontak |
| Ala-Cort | Onxal |
| Aldesleukin | Oprevelkin |
| Alemtuzumab | Orapred |
| Alitretinoin | Orasone |
| Alkaban-AQ | Oxaliplatin |
| Alkeran | Paclitaxel |
| All-transretinoic acid | Pamidronate |
| Alpha interferon | Panretin |
| Altretamine | Paraplatin |
| Amethopterin | Pediapred |
| Amifostine | PEG Interferon |
| Aminoglutethimide | Pegaspargase |
| Anagrelide | Pegfilgrastim |
| Anandron | PEG-INTRON |
| Anastrozole | PEG-L-asparaginase |
| Arabinosylcytosine | Phenylalanine Mustard |
| Ara-C | Platinol |
| Aranesp | Platinol-AQ |
| Aredia | Prednisolone |
| Arimidex | Prednisone |
| Aromasin | Prelone |
| Arsenic trioxide | Procarbazine |
| Asparaginase | PROCRIT |
| ATRA | Proleukin |
| Avastin | Prolifeprospan 20 with Carmustine implant |
| BCG | Purinethol |
| BCNU | Raloxifene |
| Bevacizumab | Rheumatrex |
| Bexarotene | Rituxan |
| Bicalutamide | Rituximab |
| BiCNU | Roveron-A (interferon alfa-2a) |
| Blenoxane | Rubex |
| Bleomycin | Rubidomycin hydrochloride |
| Bortezomib | Sandostatin |
| Busulfan | Sandostatin LAR |
| Busulfex | Sargramostim |
| C225 | Solu-Cortef |
| Calcium Leucovorin | Solu-Medrol |
| Campath | STI-571 |
| Camptosar | Streptozocin |
| Camptothecin-11 | Tamoxifen |
| Capecitabine | Targretin |
| Carac | Taxol |
| Carboplatin | Taxotere |
| Carmustine | Temodar |
| Carmustine wafer | Temozolomide |
| Casodex | Teniposide |
| CCNU | TESPA |
| CDDP | Thalidomide |
| CeeNU | Thalomid |
| Cerubidine | TheraCys |
| cetuximab | Thioguanine |
| Chlorambucil | Thioguanine Tabloid |
| Cisplatin | Thiophosphoamide |
| Citrovorum Factor | Thioplex |
| Cladribine | Thiotepa |
| Cortisone | TICE |
| Cosmegen | Toposar |
| CPT-11 | Topotecan |
| Cyclophosphamide | Toremifene |
| Cytadren | Trastuzumab |
| Cytarabine | Tretinoin |
| Cytarabine liposomal | Trexall |
| Cytosar-U | Trisenox |
| Cytoxan | TSPA |
| Dacarbazine | VCR |
| Dactinomycin | Velban |
| Darbepoetin alfa | Velcade |
| Daunomycin | VePesid |
| Daunorubicin | Vesanoid |
| Daunorubicin | Viadur |
| hydrochloride | Vinblastine |
| Daunorubicin liposomal | Vinblastine Sulfate |
| DaunoXome | Vincasar Pfs |
| Decadron | Vincristine |
| Delta-Cortef | Vinorelbine |
| Deltasone | Vinorelbine tartrate |
| Denileukin diftitox | VLB |
| DepoCyt | VP-16 |
| Dexamethasone | Vumon |
| Dexamethasone acetate | Xeloda |
| dexamethasone sodium | Zanosar |
| phosphate | Zevalin |
| Dexasone | Zinecard |
| Dexrazoxane | Zoladex |
| DHAD | Zoledronic acid |
| DIC | Zometa |
| Diodex | Gliadel wafer |
| Docetaxel | Glivec |
| Doxil | GM-CSF |
| Doxorubicin | Goserelin |
| Doxorubicin liposomal | granulocyte - colony stimulating factor |
| Droxia | Granulocyte macrophage colony stimulating |
| DTIC | factor |
| DTIC-Dome | Halotestin |
| Duralone | Herceptin |
| Efudex | Hexadrol |
| Eligard | Hexalen |
| Ellence | Hexamethylmelamine |
| Eloxatin | HMM |
| Elspar | Hycamtin |
| Emcyt | Hydrea |
| Epirubicin | Hydrocort Acetate |
| Epoetin alfa | Hydrocortisone |
| Erbitux | Hydrocortisone sodium phosphate |
| Erwinia L-asparaginase | Hydrocortisone sodium succinate |
| Estramustine | Hydrocortone phosphate |
| Ethyol | Hydroxyurea |
| Etopophos | Ibritumomab |
| Etoposide | Ibritumomab Tiuxetan |
| Etoposide phosphate | Idamycin |
| Eulexin | Idarubicin |
| Evista | Ifex |
| Exemestane | IFN-alpha |
| Fareston | Ifosfamide |
| Faslodex | IL - 2 |
| Femara | IL-11 |
| Filgrastim | Imatinib mesylate |
| Floxuridine | Imidazole Carboxamide |
| Fludara | Interferon alfa |
| Fludarabine | Interferon Alfa-2b (PEG conjugate) |
| Fluoroplex | Interleukin - 2 |
| Fluorouracil | Interleukin-11 |
| Fluorouracil (cream) | Intron A (interferon alfa-2b) |
| Fluoxymesterone | Leucovorin |
| Flutamide | Leukeran |
| Folinic Acid | Leukine |
| FUDR | Leuprolide |
| Fulvestrant | Leurocristine |
| G-CSF | Leustatin |
| Gefitinib | Liposomal Ara-C |
| Gemcitabine | Liquid Pred |
| Gemtuzumab ozogamicin | Lomustine |
| Gemzar | L-PAM |
| Gleevec | L-Sarcolysin |
| Lupron | Meticorten |
| Lupron Depot | Mitomycin |
| Matulane | Mitomycin-C |
| Maxidex | Mitoxantrone |
| Mechlorethamine | M-Prednisol |
| Mechlorethamine | MTC |
| Hydrochlorine | MTX |
| Medralone | Mustargen |
| Medrol | Mustine |
| Megace | Mutamycin |
| Megestrol | Myleran |
| Megestrol Acetate | Iressa |
| Melphalan | Irinotecan |
| Mercaptopurine | Isotretinoin |
| Mesna | Kidrolase |
| Mesnex | Lanacort |
| Methotrexate | L-asparaginase |
| Methotrexate Sodium | LCR |
| Methylprednisolone | |

As used herein, the term “tumor” refers to all neoplastic cell growth and proliferation, whether malignant or benign, and all pre-cancerous and cancerous cells and tissues. For example, a particular cancer may be characterized by a solid tumor mass or a non-solid tumor. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture, or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site. The peptides of the invention are capable of inducing apoptosis in tumor cells and reducing tumor cell growth. The peptides of the invention (or nucleic acids encoding them) can be administered locally at the site of a tumor (e.g., by direct injection) or remotely. The peptides of the invention can induce cell death in circulating tumor cells (CTC) in a subject, e.g., by administering the peptides or encoding nucleic acids intravenously. Furthermore, the peptides of the invention can prevent or reduce onset of metastasis to other tissues, e.g., to the bone.

As used herein, the term “signaling” and “signaling transduction” represents the biochemical process involving transmission of extracellular stimuli, via cell surface receptors through a specific and sequential series of molecules, to genes in the nucleus resulting in specific cellular responses to the stimuli.

The teen “vector” is used to refer to any molecule (e.g., nucleic acid, plasmid, or virus) usable to transfer coding sequence information (e.g., nucleic acid sequence encoding the HYD1 peptide or a fragment or variant thereof), such as to a host cell. A vector typically includes a replicon in which another polynucleotide segment is attached, such as to bring about the replication and/or expression of the attached segment. The term includes expression vectors, cloning vectors, and the like. Thus, the term includes gene expression vectors capable of delivery/transfer of exogenous nucleic acid sequences into a host cell. The term “expression vector” refers to a vector that is suitable for use in a host cell (e.g., a patient's cell, tissue culture cell, cells of a cell line, etc.) and contains nucleic acid sequences which direct and/or control the expression of exogenous nucleic acid sequences. Expression includes, but is not limited to, processes such as transcription, translation, and RNA splicing, if introns are present. Nucleic acid sequences can be modified according to methods known in the art to provide optimal codon usage for expression in a particular expression system. The vector may include elements to control targeting, expression and transcription of the nucleic acid sequence in a cell selective manner as is known in the art. It should be noted that often the 5′UTR and/or 3′UTR of the gene may be replaced by the 5′UTR and/or 3′UTR of the expression vehicle. The vector can include a promoter for controlling transcription of the exogenous material and can be either a constitutive or inducible promoter and/or a tissue-specific promoter, to allow selective transcription. The expression vector can also include a selection gene.

A “coding sequence” is a polynucleotide sequence that is transcribed into mRNA and/or translated into a polypeptide. The boundaries of the coding sequence are determined by a translation start codon at the 5′-terminus and a translation stop codon at the 3′-terminus.

A coding sequence can include, but is not limited to, mRNA, cDNA, and recombinant polynucleotide sequences. Variants or analogs may be prepared by the deletion of a portion of the coding sequence, by insertion of a sequence, and/or by substitution of one or more nucleotides within the sequence. Techniques for modifying nucleotide sequences, such as site-directed mutagenesis, are well known to those skilled in the art (See, e.g., Sambrook et al., Molecular Cloning: A Laboratory Manual, Second Edition, 1989; DNA Cloning, Vols. I and II, D. N. Glover ed., 1985). Optionally, the polynucleotides of the present invention, and composition and methods of the invention that utilize such polynucleotides, can include non-coding sequences.

The term “operably-linked” is used herein to refer to an arrangement of flanking control sequences wherein the flanking sequences so described are configured or assembled so as to perform their usual function. Thus, a flanking control sequence operably-linked to a coding sequence may be capable of effecting the replication, transcription and/or translation of the coding sequence under conditions compatible with the control sequences. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a polypeptide will typically have its own operably-linked promoter sequence.

The invention includes administration of HYD1 peptides or other CD44-binding agent to a malignant cell in vitro or in vivo through administration (transfection) of a nucleic acid encoding the agent (e.g., the HYD1 peptide) in a viral or non-viral vector. The terms “transfection” and “transformation” are used interchangeably herein to refer to the insertion of an exogenous polynucleotide into a host cell, irrespective of the method used for the insertion, the molecular form of the polynucleotide that is inserted, or the nature of the cell (e.g., prokaryotic or eukaryotic). The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome.

As used herein, the term “pharmaceutically acceptable salt or prodrug” is intended to describe any pharmaceutically acceptable form (such as an ester, phosphate ester, salt of an ester or a related group) of a compound of the invention, which, upon administration to a subject, provides the mature or base compound (e.g., a HYD1 peptide). Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The terms “link” or “join” refers to any method known in the art for functionally connecting peptides, including, without limitation, recombinant fusion, covalent bonding, disulfide bonding, ionic bonding, hydrogen bonding, and electrostatic bonding.

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state. Thus, isolated peptides in accordance with the invention preferably do not contain materials normally associated with the peptides in their in situ environment.

As used in this specification, the singular forms “a”, “an”, and “the” include plural reference unless the context clearly dictates otherwise. Thus, for example, a reference to “a cell” includes one or more cells. A reference to “a peptide” includes one or more such peptide, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover

Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

### Materials and Methods

Cell Culture.

NCI-H929, RPMI-8226 and HS-5 cells were obtained from the American Type Culture Collection (Rockfield, Md.). 293FT cells were obtained from Invitrogen and grown in Iscove's DMEM (Cellgro,) supplemented with 10% FBS. Chemical Reagents, Antibodies, and Peptides. 5-Chloromethylfluoroscein diacetate was purchased from Invitrogen (Carlsbad Calif.). Melphalan, mitoxantrone dihydrochloride, and N-acetyl-L-cysteine were purchased from Sigma-Aldrich (St. Louis Mo.). Anti-α4 integrin (clone P4G9) antibody was purchased from Abcam (San Francisco Calif.). Anti-CD29 (4B7R) antibody was purchased from Novus Biologicals (Littleton, Colo.). FITCconjugated anti-integrin 62 7 (FIB504) was purchased from Biolegend (San Diego Calif.). HYD1 (kikmviswkg) was synthesized by Bachem (San Diego Calif.) and FAM conjugated HYD1 (FAM-kikmviswkg) were synthesized by Global Peptides (Fort Collins, Colo.).

Selection of a Drug-Resistant Cell Line.

A HYD1 resistant cell line was developed as tool for delineating determinants of resistance and sensitivity to HYD1 induced cell death. NCI-H929 cells were exposed to increasing concentrations of HYD1 for 24 weeks. The cells were named H929-60 and are maintained in media containing 60 ug/ml HYD1, once a week for 24 hours.

Cell Death Analysis. After treatment with HYD1, cells were washed with PBS and incubated with 2 nM TO-PRO-3 iodide for 45 minutes. The cells were analyzed for fluorescence with the use of a FACSCalibur (BD Biosciences, San Jose, Calif.).

Measurement of Δψm.

After treatment, cells were incubated for 15 minutes with 15 nM of 3,3′-dihexyloxacarbocyanine iodide (Invitrogen). Cells were washed and resuspended in PBS, and the loss of mitochondrial membrane potential was measured using FACScan.

ATP Measurement.

Treated cells were lysed in radioimmunoprecipitation assay buffer (Millipore, Billerica, Md.), and ATP concentrations were measured using the ENLITEN ATP bioluminescence detection kit per manufacturer's instructions (Promega, Madison, Wis.). Samples were later normalized to the protein content of the lysates.

Confocal Microscopy.

To assess whether (a) HYD1 bound the cell surface and (b) whether binding of HYD1 was reduced in the resistant cell line FAM-HYD1 was used to image peptide binding in the parental and resistant cell line by confocal microscopy. A 35 mm glass bottom microwell dishes (Mattek cultureware, Ashland, Mass.) were plated with 10 uL Cell-tak (BD biosciences) per manufacture's instructions. Media containing 1 uM Alexa Fluor 594 wheat germ agglutinin (WGA) (Invitrogen) and 20 uM Hoechst 33342 (Invitrogen) was placed back in the plates and the samples were incubated for 30 minutes. After 30 minutes, the cells were washed and treated with media containing 6.25 μg/ml FAM conjugated HYD1 for 10 minutes. Samples were immediately viewed with a Leica DMI6000 confocal microscope (Leica Microsystems, Germany). Gain, offset, and pinhole setting were identical for all samples within the treatment group.

Surface Expression of Integrins.

To determine cell surface expression of integrins, cells were treated with a primary antibody for 45 minutes on ice. Cells were then washed and treated with a FITC conjugated secondary antibody for 45 minutes on ice. Cells were washed again and the fluorescence analyzed using a FACScan (BD Biosciences).

Reverse Transcriptase Polymerase Chain Reaction (Rt-PCR).

Rt-PCR was used to determine whether the decrease in a4 integrin protein levels in the resistant cell line was due to decreased transcription. RNA was extracted from log growth cells with RNeasy columns (Qiagen, Valencia, Calif.) per manufacturer's instructions. First-strand cDNA synthesis was carried out with the Quantitect Probe RT-PCR kit (Qiagen, Valencia, Calif.) per manufacturer's instructions. Real time PCR primers for alpha 4 integrin were obtained from Applied Biosystems (Carlsbad, Calif.). The gene-expression level was normalized using the endogenous control gene GAPDH. Real-time PCR reactions were performed using ABI 7900 Sequence Detection System (Applied Biosystems).

Cell Adhesion to ECM Proteins and Stroma.

Cell adhesion assays were performed to determine whether the resistant cell line demonstrated reduced adhesive capacity. Briefly, cells were pre-incubated with various antibodies for 30 minutes. Cells were then allowed to attach to 50 uL of 40 ug/mL FN (Roche, Indianapolis, Ind.) or 10 ug/mL VCAM-1 (Fisher Scientific, Pittsburgh, Pa.) for two hours. After 2 hours, cell adhesion was detected by crystal violet staining as previously described (17). For stromal adhesion, 10,000 HS-5 cells were incubated overnight on immunosorb 96 well plates (Nunc, Denmark). H929 and H929-60 cells were incubated with 1 uM of CMFDA (Invitrogen) for 30 minutes, washed and incubated for 45 minutes to allow unbound dye to diffuse out of the cells. Intensity was read on a fluorescence plate reader (9).

Transfection of shRNAs.

ShRNA targeting strategies were used to determine whether a4 integrin expression was causally related to HYD1 induced cell death. α4 (TRCN0000029656) and in (TRCN0000029645) shRNA and non-silencing clone sets were purchased from Open Biosystems, Huntsville, Ala. and transfected into a lentivirus using the BLOCK-IT Lentiviral Pol II miR RNAi expression system (Invitrogen). After the viral supernatant was collected, 500,000 myeloma cells were infected with a 250 uL viral supernatant/750 uL media solution and 5 ug/mL polybrene (Sigma) for 24 hours. After 24 hours, the solution was removed and replaced with fresh media. At 72 hours of infection, 1 ug/mL puromycin (Invitrogen) was added to 8226 cells to allow for the selection of a stable population of cells. For the H929 cell line transient infections were used to reduce integrin expression.

Biotin-HYD1 Pull-Down Assay.

To identify HYD1 interacting proteins biotin conjugated HYD1 was used as bait as previously described (15). Briefly, 100 million H929 or the resistant variant H929-60 were washed once in PBS and then in AP buffer. Samples were spun at 15000 XG for minutes at 4 degrees and the supernatant was removed and membranes were pelleted at 100,000Xg for 15 minutes. Membrane pellets were solubilized in AP buffer containing 0.2% NP40 and protein was quantified using BCA reagents (Pierce, Rockford, Ill.). Five hundred ug of Biotin-HYD1 was bound to 30 ul of UltraLink Neutra Avidin Plus beads (Pierce) for one hour in a buffer containing 0.5 mM KCl, 0.3 mM KH2PO₄, 27.6 mM NaCl and 1.6 mM Na₂HPO₄, pH 7.4. After one hour, the beads were washed twice in AP buffer and 50 ug of membrane extracts were added to a total volume of 500 μl and incubated with beads overnight at 4 degrees. The beads were washed five times in AP buffer containing 0.2% NP40 and samples were suspended in SDS-PAGE sample buffer and bound proteins were resolved by SDSPAGE.

Isolation of CD138 Positive and Negative Populations Derived from Multiple Myeloma Specimens.

To determine whether HYD1 was active in primary patient specimens 7 newly diagnosed and 7 relapsed specimens were obtained. Myeloma patients were consented through the Total Cancer Care tissue banking protocol per IRB regulations. Mononuclear cells were separated from human blood through the use of Ficoll-Paque PLUS (GE Healthcare, UK).

After separation, CD 138 positive cells were sorted using 25 MS MACS Separation Columns (Miltenyi Biotec, Germany) and CD 138 microbeads (Miltenyi) per manufacturer's instructions. For each specimen obtained a4 integrin surface expression was determined by FACS analysis and HYD1 induced cell death was determined by Topro-3 staining and FACS analysis in the CD138 positive and negative fraction.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### HYD1 is More Active in MM Cells Compared to Normal Cells

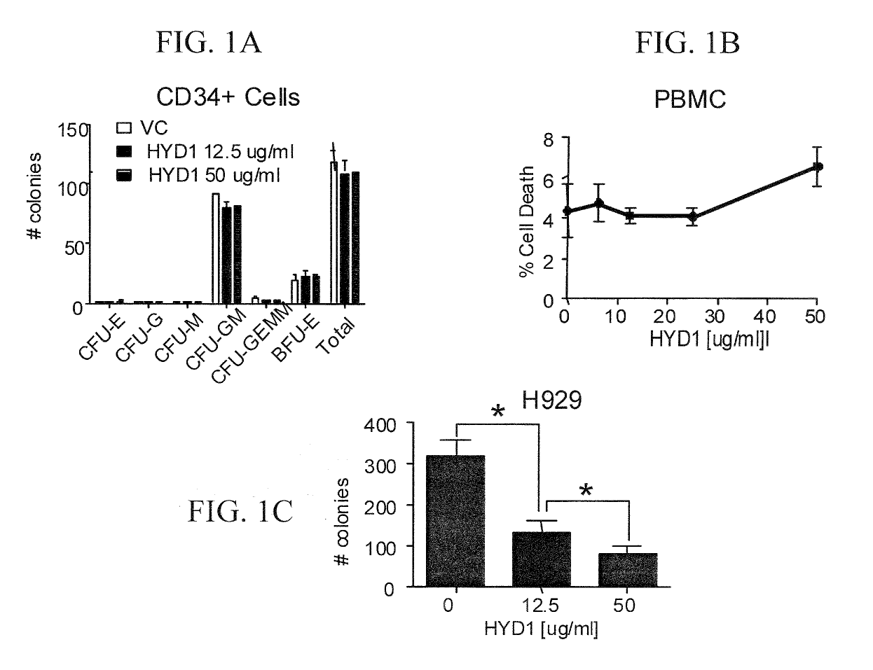

A colony forming assay was used to determine whether HYD1 induced cell death in normal hematopoietic cells. CD34⁺ hematopoietic progenitor cells were isolated from peripheral blood and treated for 2 hrs with HYD1 (12.5 and 50 μg/ml) and then plated in a methylcellulose media supplemented with growth factors supporting myeloid and erythroid colonies. Colonies were counted on day 12 post-plating. As shown in FIG. 1A, HYD1 did not inhibit colony formation of normal CD34⁺ cells. In addition, the inventors evaluated the toxicity of HYD1 in normal peripheral blood mononuclear cells (PBMC). As seen in FIG. 1B, six hours treatment with increasing concentration of HYD1 did not induce cell death up to doses of 50 μg/ml in PBMC. Finally as shown in FIG. 1C, and consistent with Topro-3 staining, HYD1 did inhibit colony formation of H929 cells at doses ranging from 12.5-50 ug/ml HYD1. Together, the data indicate that HYD1 targets MM cells preferentially when compared to normal hematopoietic cells.

### Example 2

### HYD1 Induces Necrotic Cell Death

HYD1-induced cell death is necrotic in nature as shown by: (a) a decrease in mitochondrial membrane potential (Δψₘ); (b) a loss of total cellular ATP, and; (c) an increase in reactive oxygen species (ROS) production. Moreover, HYD1 treatment does not result in apoptotic cell death as it did not trigger the activation of caspases or the release of apoptosis-inducing factor (AIF) and Endonuclease G (Endo G) from the mitochondria, nor did it induce double-stranded DNA breaks. HYD1 did initiate autophagy in cells; however, autophagy was found to be an adaptive response contributing to cell survival rather than the cause of cell death. The inventors were further able to show that N-acetyl-L-cysteine (NAC), a thiol containing free radical scavenger, partially protects MM cells from HYD1-induced death (Nair et al. 2009).

### Example 3

### HYD1 Shows Activity as a Single Agent in the SCID-Hu In Vivo Model

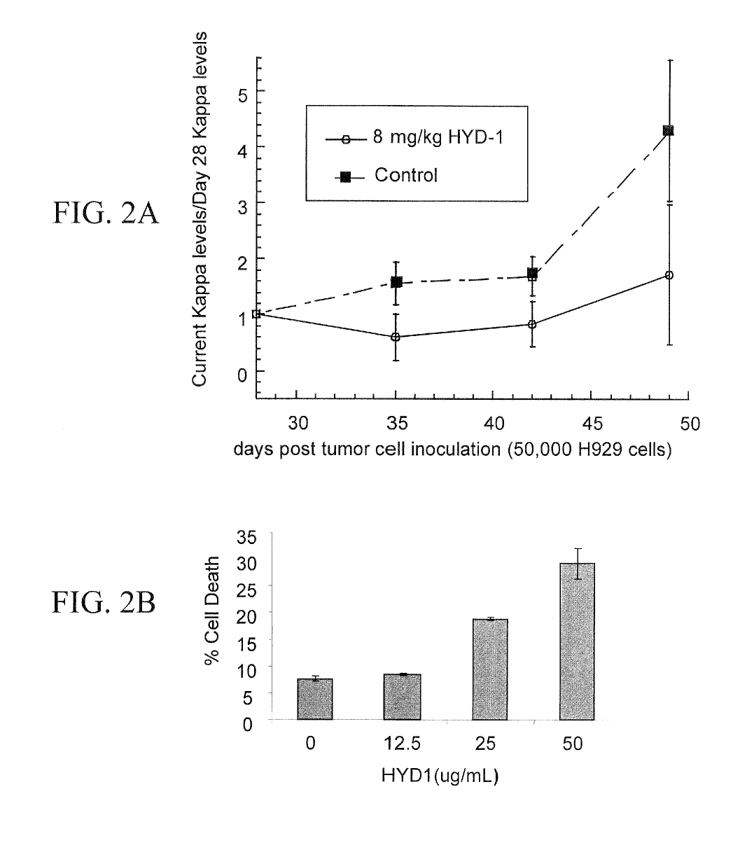

The inventors used the SCID-hu model to determine whether HYD1 demonstrates anti-tumor activity in vivo. The SCID-hu model consists of implanting human fetal bone into the mammary mouse fat pad of SCID mice. The myeloma cells are subsequently injected directly into the bone and myeloma cells will engraft only in the area of the human bone. Human paraprotein can be measured in the mouse sera and levels represent a good marker for evaluating tumor burden and response to chemotherapy. Circulating kappa levels were measured on day 28 (baseline reading before peptide treatment), 35, 42 and 49 by ELISA, and tumor burden was determined by calculating the kappa levels on day X divided by the baseline kappa levels recorded on day 28 for each mouse. Shown in FIG. 2A is the antitumor response of H929 engrafted tumor when mice were given 8 mg/kg intraperitoneal (i.p.) injections daily for 21 days starting on day 28. Mice treated with HYD1 showed a reduction in tumor burden compared to control mice (p<0.05, repeated measures test n=5 control, n=4 HYD1 treated mice). Mice were humanely euthanized at day 28 as several control mice had tumor protruding from the bone implant. In these experiments, no overt toxicity or weight loss was noted in HYD1 treated animals. HYD1 does induce cell death in 5TGM1 cells, a mouse myeloma cell line, indicating that the lack of overt toxicity cannot be explained by failure of HYD1 to interact with the mouse homolog (FIG. 2B). Taken together, these data indicate that HYD1 retains anti-tumor activity in vivo and does not induce overt signs of toxicity. The inventors anticipate that because HYD1 induces cell death independent of apoptosis and blocks cell adhesion mediated drug resistance to standard therapy that the most dramatic in vivo activity will be realized when combined with standard therapy. To this end, the inventors have preformed synergy experiments in vitro and determined that Doxorubicin shows the greatest levels of synergy with HYD1.

### Example 4

### Reducing α4 and β1 Integrin Expression Confers Partial Resistance to HYD1 Induced Cell Death

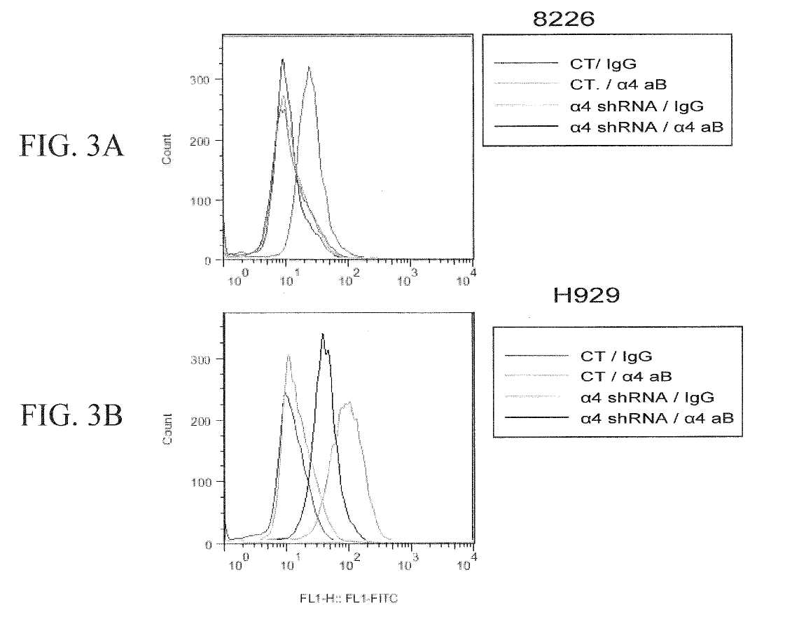

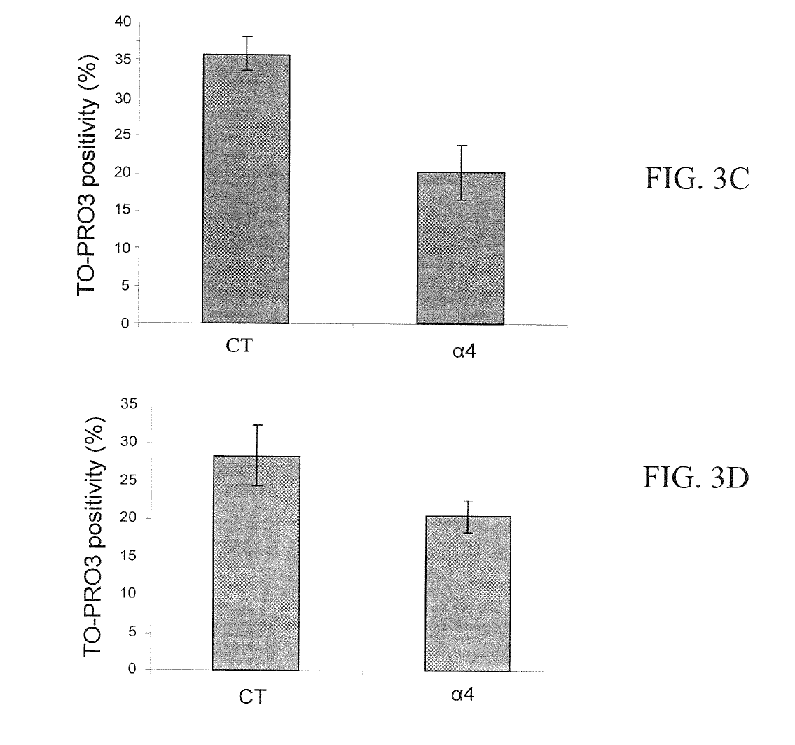

Currently, 11 known α binding partners for β1 integrin have been identified. To aid in the delineation of markers of sensitivity and resistance to HYD1, the inventors developed a HYD1 drug resistant isogenic MM cell line by chronically exposing H929 cells to increasing doses of HYD1. The genotypic and phenotypic characterization of that cell line showed reduced α4 and β1 integrin expression and ablated α4β1 mediated adhesion to the extracellular matrix fibronectin. Thus, the inventors initially sought to determine whether α4 and/or β1 expression is required for HYD1 mediated cell death. As shown in FIGS. 3A-3D, reducing α4 integrin levels in H929 and 8226 cells using shRNA partially blocked HYD1 induced cell death. Similar levels of resistance were observed by reducing the levels of β1 integrin. However, induction of cell death in MM cell lines was not observed with a blocking α4 or β1 integrin or RGD peptide. Furthermore, the observation that reducing a4 integrin did not abrogate HYD1 induced cell death suggests that additional adhesion/cell surface moieties may be involved in HYD1 mediated cell death. The observation that blocking integrin antibodies did not recapitulate the phenotype indicates that the peptide is not simply blocking integrin mediated binding of fibronectin or VCAM1.

### Example 5

### Biotin-HYD1 Interacts with CD44

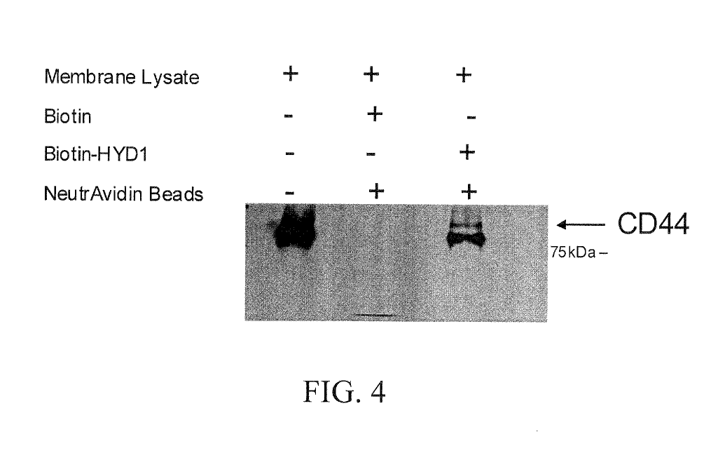

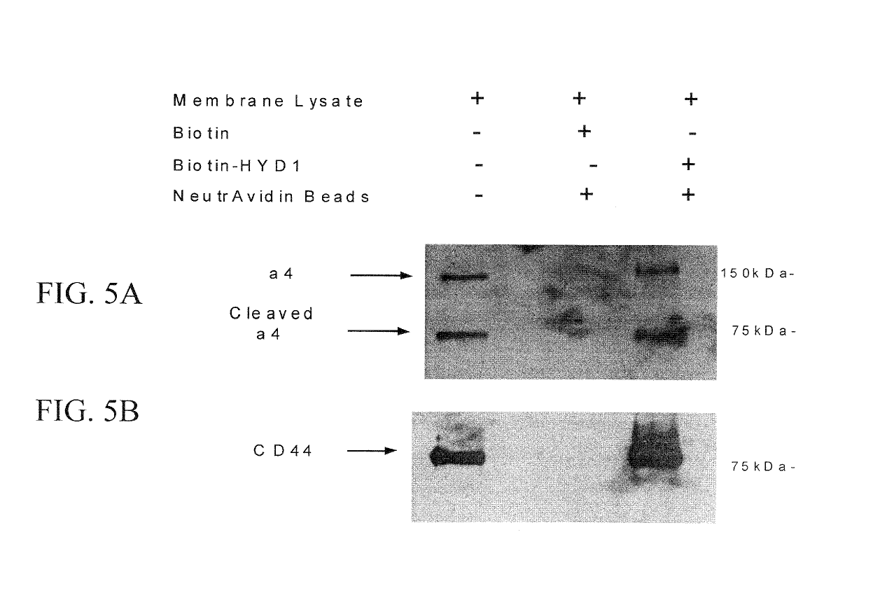

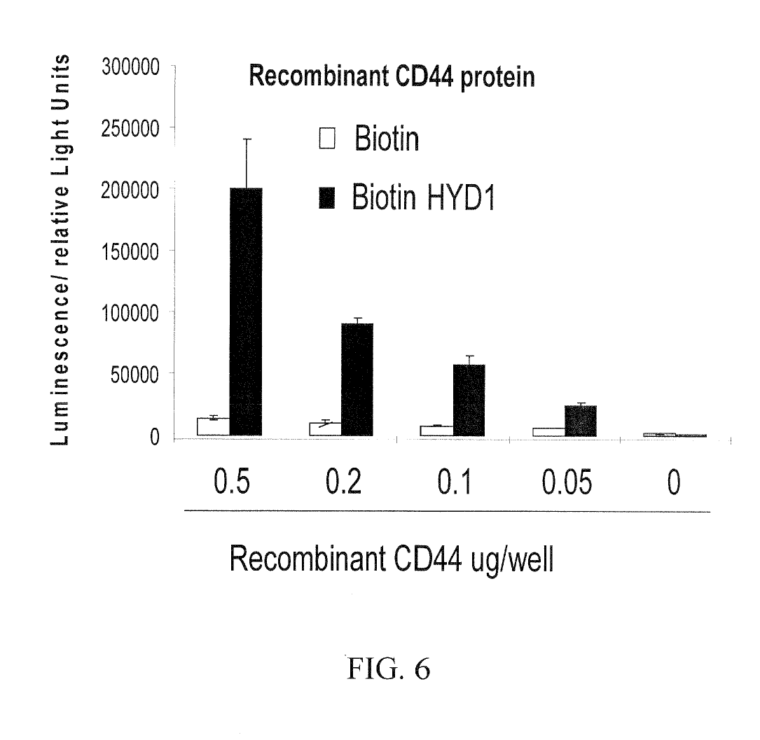

The inventors used biotin-HYD1 as bait to pulldown binding complexes contained within membrane extracts of H929 MM cells. The pull down assay was directly coupled with an unbiased Mass-Spec analysis to identify HYD1 binding partners. Before performing these studies, the inventors confirmed that biotinylation of HYD1 did not inhibit the bioactivity of the compound, as the IC₅₀ value for biotin-HYD1 was slightly decreased in H929 cells. NeutrAvidin beads were used to reduce non-specific binding. The control sample consisted of incubating the membrane extract with biotin and subsequently subjecting the sample to NeutraAvidin beads similar to the biotin-HYD1 sample. In the 30 ug of membrane extract, the only cell surface protein that the inventors identified that was specific for the biotin-HYD1 sample was CD44. The binding experiment was repeated using 300 ug of membrane extract. In the scaled up reaction, α4 integrin, β1 integrin, NCAM and syndecan-1 were indentified by Mass-Spec analysis. As shown in FIG. 4, Western blot analysis was used to confirm that biotin-HYD1 and not biotin interacted with CD44 (antibody used is a pan CD44 antibody). The inventors next determined whether α4 integrin could be detected by western blot analysis. As shown in FIG. 5A, the inventors were able to show that α4 was present in the complex; however, stripping the blot and re-probing the membrane revealed that the Biotin-HDY1 complex contains more CD44 compared to a4 integrin (see FIG. 5B). To determine whether CD44 was indeed a direct binding partner of CD44 the inventors used recombinant CD44 and an ELISA as a readout of binding. The recombinant CD44 protein (purchased from Abnova) corresponds to Isoform 4 on Swiss Prot. The amino acid sequence is missing 224-266 and 223 is substituted S for T relative to the longest CD44 variant referred to as epican. As shown in FIG. 6, the inventors were able to capture CD44 in biotin-HYD1 NeutraAvidin coated 96-well plates. Again, Biotin-coated NetraAvidin showed relatively no binding of CD44. Collectively, these data indicate that CD44 is the likely direct binding target of CD44. Both U226 and H929 cells which are relatively sensitive to HYD1 are reported to express the CD44s (standard form) and the variant forms CD44v3 and CD44v9.

### Example 6

### Activity of HYD1 in MM Primary Patient Specimens HYD1 Correlates with α4 And CD44 Expression

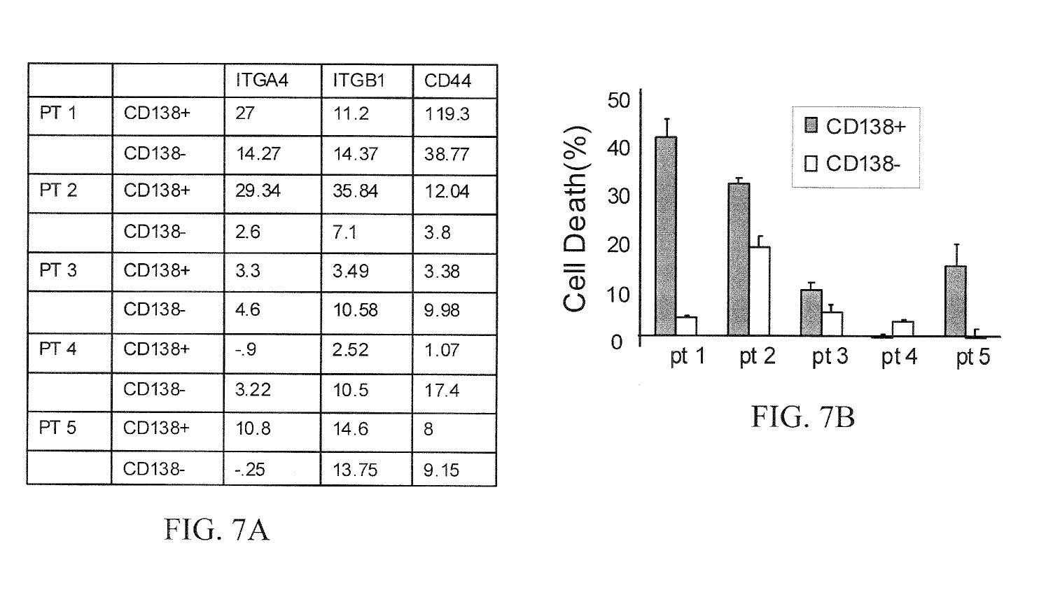

The inventors propose that the development of biomarkers of response should be done in the early stages of validation of targets and novel drug candidates. In addition to α4 integrin being a determinant of resistance, the inventors have identified CD44 as the putative binding target of HYD1. The inventors anticipate that delineation of the binding complexes associated with α4 and CD44 in the presence and absence of HYD1 will allow for better understanding of the downstream signaling that is required for cell death. However, the inventors have defined two biomarkers that are anticipated in which expression will be required for a positive HYD1 response (CD44 and α4 integrin). To this end, a pilot study was performed in MM patients. As shown in FIG. 7A, CD44, α4 and β1 integrin expression is more abundant in the CD138 positive myeloma cells compared to the negative population. Furthermore, the potency of HYD1 correlated with the expression of α4 integrin and CD44 (FIG. 7B) which supports expression of CD44 and/or a4 integrin as a predictor of sensitivity to HYD1 induced cell death.

### Example 7

### HYD1 Exhibits Synergistic Growth Inhibition with Other Anti-Cancer Agents in a Myeloma Cell Line

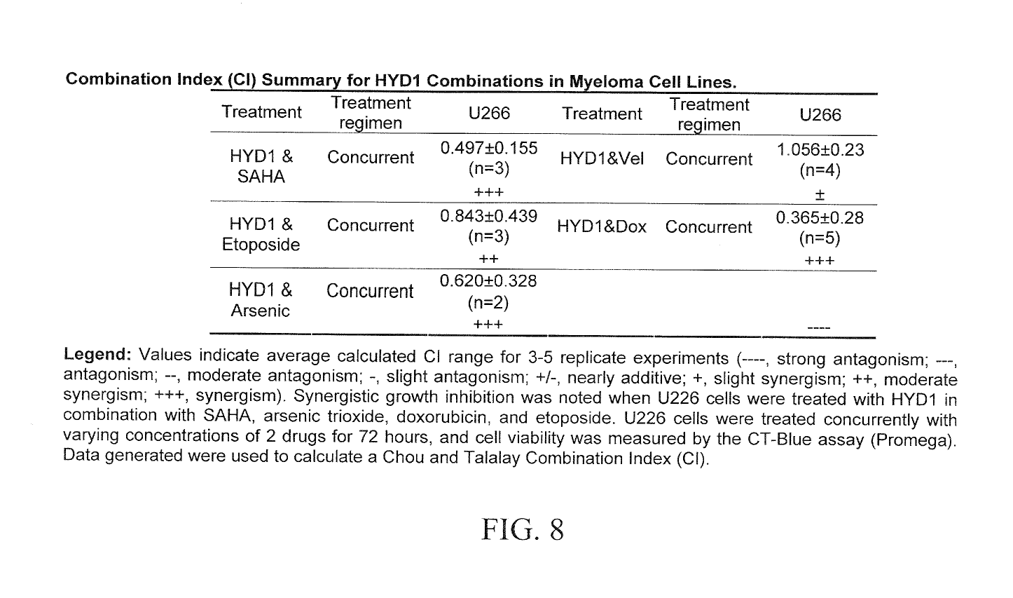

As shown in the table of FIG. 8, a combination cytotoxicity assay was performed in vitro using U226 cells. Doxorubicin was shown to be the most synergistic in combination with HYD1.

### Example 8

### H929-60 Cells are Resistant to HYD1 Induced Cell Death but do Not Show Cross-Resistance to Other Active Myeloma Agents

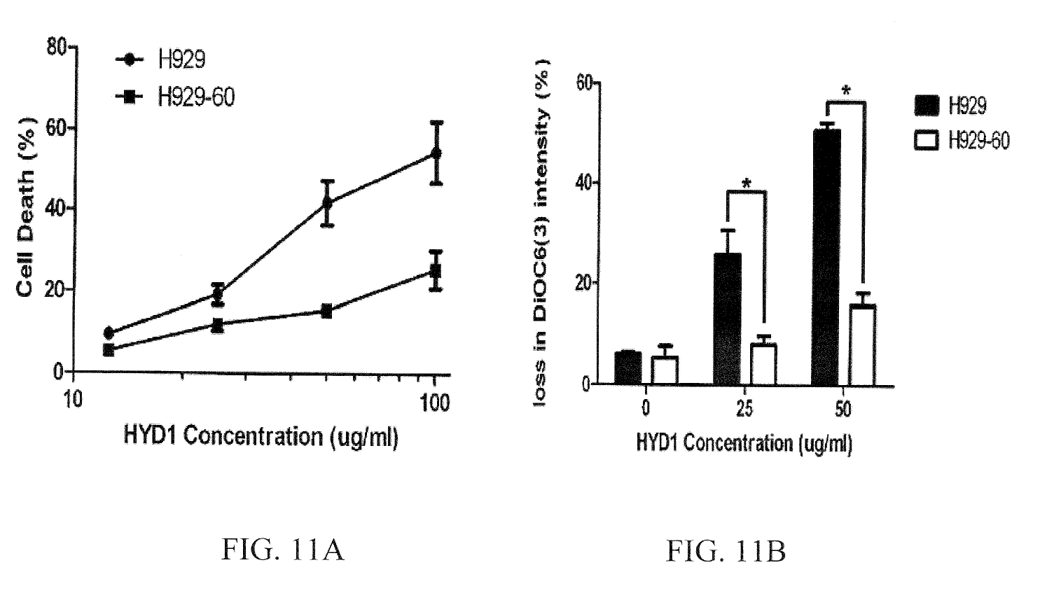

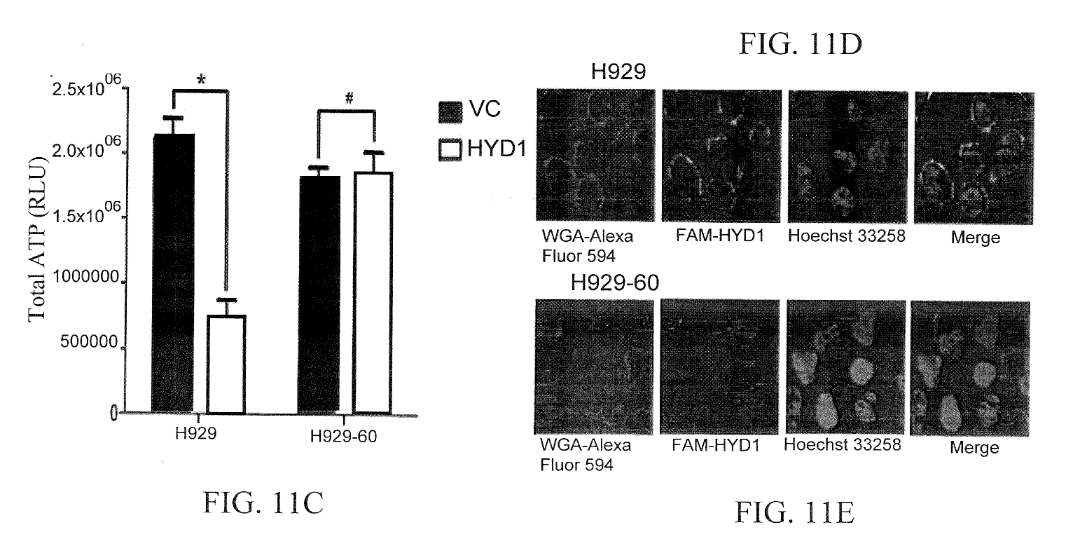

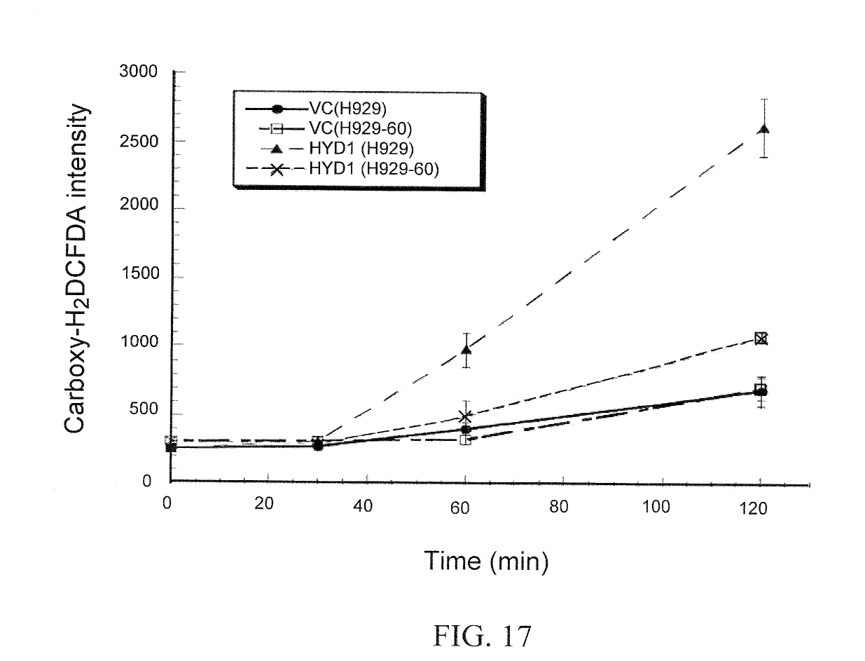

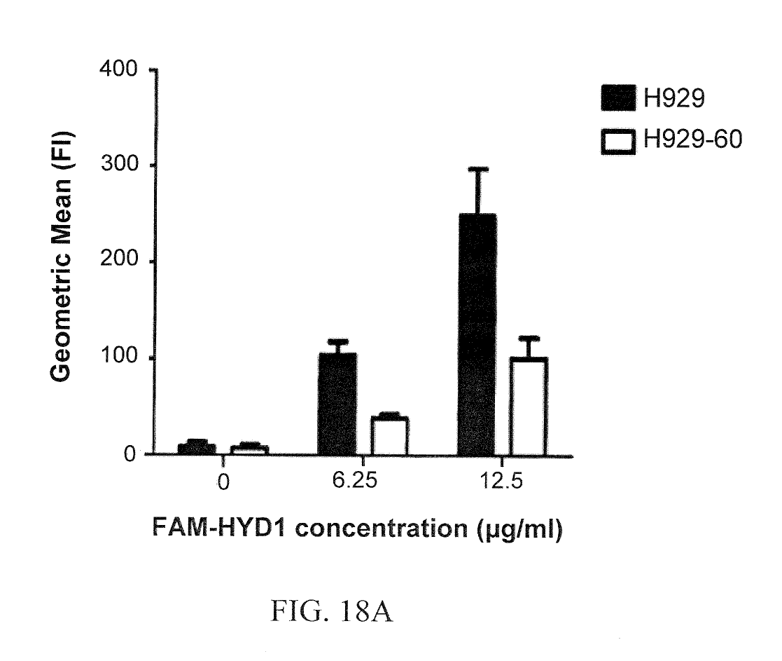

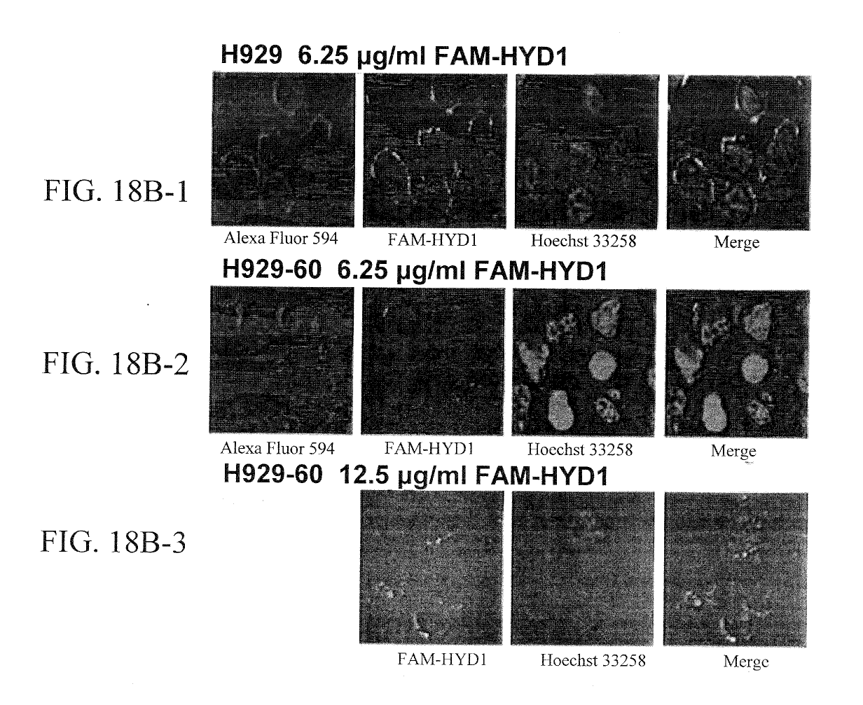

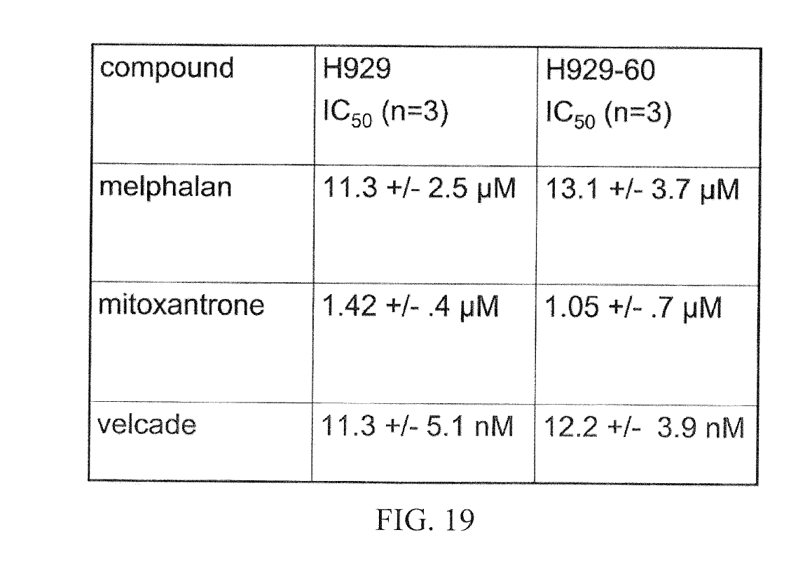

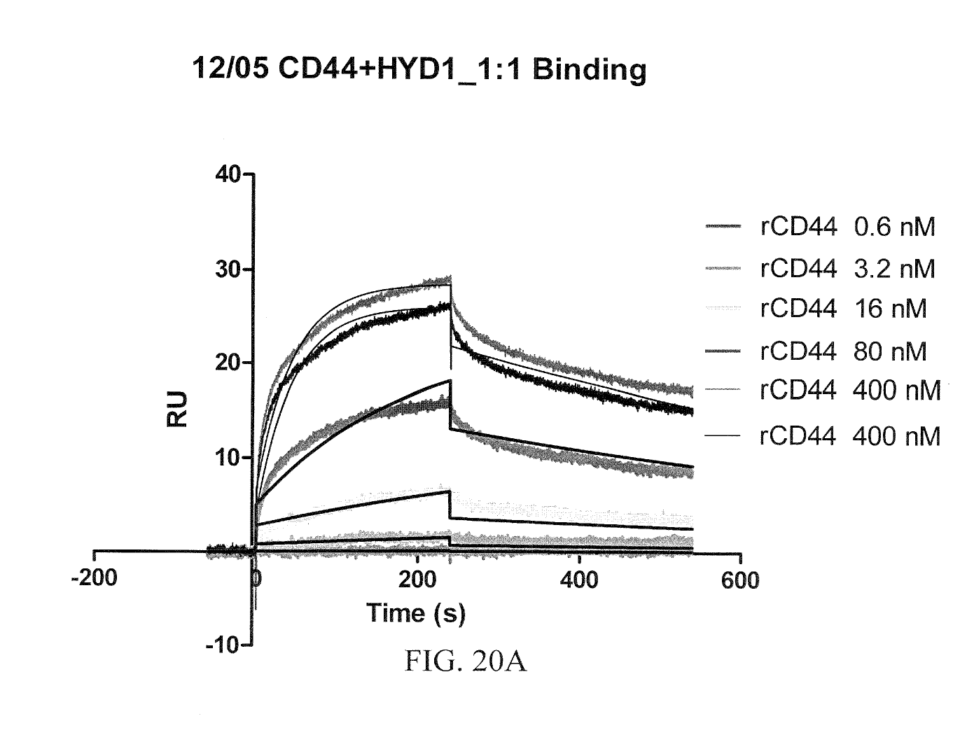

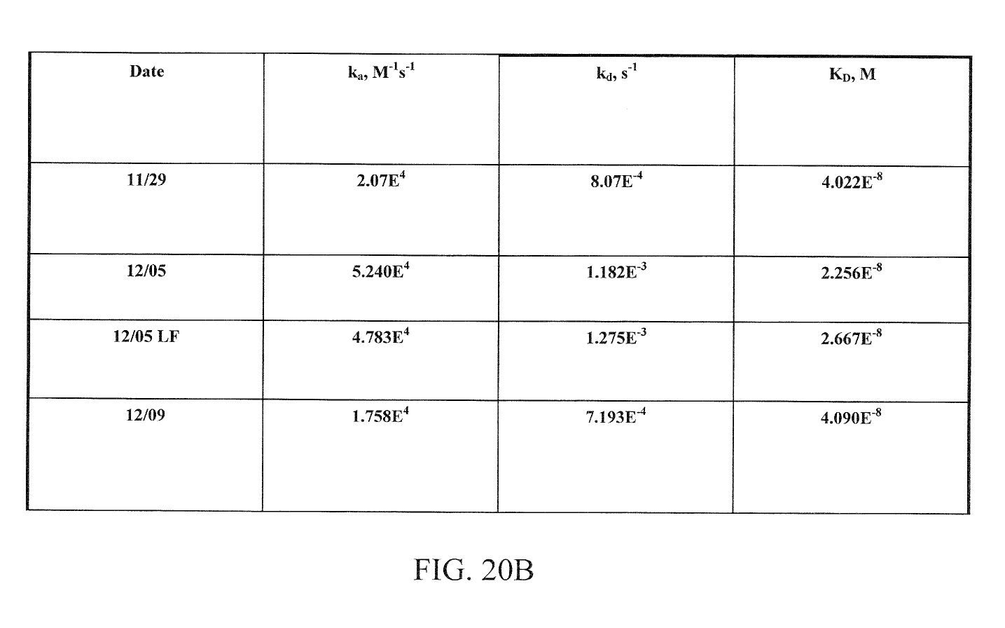

To identify determinants of resistance towards HYD1, the inventors developed an isogenic resistant H929 cell line variant by chronically exposing H929 cells to increasing concentrations of HYD1 until a drug resistant variant emerged. As shown in FIG. 11A, the H929-60 acquired resistant cell line is significantly resistant to HYD1 induced cell death (p<0.05 ANOVA) when compared to the parental H929 cells as measured by TOP-RO 3 positivity and FACS analysis. HYD1 induced cell death was previously characterized by the loss of mitochondrial membrane potential, ATP depletion, and an increase in reactive oxygen species (ROS). To determine whether the acquisition of resistance occurred upstream or downstream of mitochondria dysfunction the mitochondria membrane potential, ATP levels and ROS levels were compared following HYD1 treatment in the resistant (H929-60) and sensitive parental cell line (H929). H929-60 cells were shown to be resistant to the loss of mitochondrial membrane potential (FIG. 11B) and failed to show a reduction in ATP levels following HYD1 treatment (FIG. 11C). Finally, ROS levels were reduced following HYD1 treatment in the resistant cell line compared to the parental cell line (FIG. 17). The inventors utilized a FAM conjugated HYD1 peptide to determine whether the H929 resistant variant demonstrated a reduction in binding of FAM-HYD1 to the cell membrane. As shown in FIGS. 11D-11E, FAM-HYD1 localizes to the plasma membrane in the parental cell line. Furthermore, the localization of FAM-HYD1 was not evenly distributed across the cell membrane, but rather FAM-HYD1 demonstrated punctuated staining in the parental cell line, suggesting potential clustering of the binding target. H929-60 cells treated with 6.25 ug/ml FAM-HYD1, demonstrated a 2.7-fold reduction in FAM-HYD1 binding relative to the parental cell line as determined by FACS analysis (FIGS. 18A, 18B-1, 18B-2, and 18B-3). Collectively, these data indicate that the mechanism causative for resistance towards HYD1 occurs upstream of mitochondrial dysfunction and generation of ROS and that the resistant mechanism is likely the result of qualitative or quantitative changes in the HYD1 binding complex located on the cell membrane. Selection of acquired resistance in a population of cells (rather than clonal selection) often leads to multiple mechanisms of resistance and a phenotype that confers resistance to multiple agents (18). However, the inventors hypothesized that since HYD1 induces necrotic cell death, selection with HYD1 would not result in a phenotype which conferred resistance to other agents commonly used to treat myeloma. To address this question, the inventors compared the IC50 values of H929-60 and the parental H929 cells to the alkylating agent, melphalan, the topoisomerase II inhibitor, mitoxantrone, and the proteasome inhibitor, Velcade. As predicted, H929-60 cells were not resistant to other classes of agents known to induce apoptosis commonly used to treat multiple myeloma (see table in FIG. 19). These data further support the potential advantage of targeting necrosis in combination with inducers of apoptosis for the treatment of multiple myeloma, as cross-resistance between these two agents is unlikely to occur during the course of drug treatment.

### Example 9

### Acquisition Towards Resistance to HYD1 Results in Reduced Expression of α4, β1 Integrin and Ablated Functional Binding to Fibronectin and VCAM-1

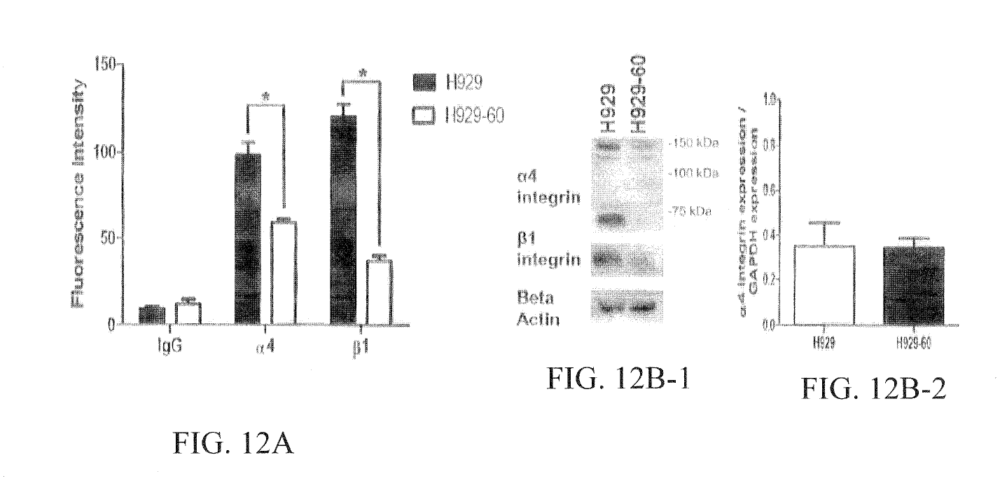

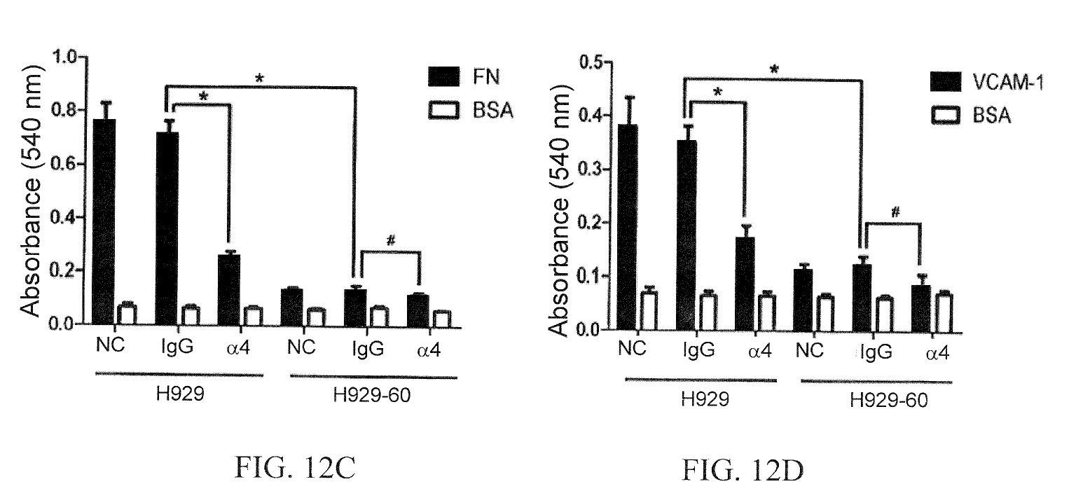

To determine whether the acquisition of resistance correlated with quantitative changes in integrin expression the inventors screened multiple a integrin sub-units (α4, α5, αV and α6 integrin data not shown) that are commonly expressed in hematopoietic cells and determined that α4β1 integrin was the most abundant integrin expressed on the parental cell line and expression was reduced in the resistant cell line (See FIG. 12A). Using FACS analysis, the inventors determined that α4 integrin was found to be reduced by 1.8 fold. As shown in FIGS. 12B-1 and 12B-2, the reduction at the cell surface corresponded to reduced protein expression using a whole cell whole cell lysate preparation. Interestingly, when examining the whole cell lysate the most dramatic decrease was the cleaved form of α4 integrin. In some cell types, the mature 150 kD a4 integrin is cleaved into a non-disulfide linked 80 and 70 kD fragment. For example, activation of T-cells was previously reported to correlate with increased cleavage of the mature α4 integrin (19). However, the cleavage of α4 integrin was found to not alter the adhesive properties of VLA-4 integrin to fibronectin or VCAM1(20). The attenuation of protein expression is post-transcriptionally regulated as the parental and resistant cell line demonstrated equal levels of α4 mRNA (FIGS. 12B-1 and 12B-2). Since β1 integrins require inside out activation in order to be competent to bind ligand, expression does not necessarily directly correlate with functional adhesion. Thus, the inventors next sought to determine whether a reduction in the expression of VLA-4 integrin resulted in a functional reduction in adhesion to extracellular matrices. To this end, the inventors compared the levels of adhesion of the sensitive and resistant cell line to fibronectin and the more specific ligand for α4 integrin, VCAM-1. In FIGS. 12C and 12D, H929-60 cells demonstrated a dramatic reduction in the binding to FN and VCAM-1. An α4 integrin blocking was used as a positive control for blocking the parental cell line to FN and V CAM-1.

### Example 10

### Reducing the Expression α4 and β1 Integrins is Causative for Resistance to HYD1 Induced Cell Death in H929 and 8226 Multiple Myeloma Cells

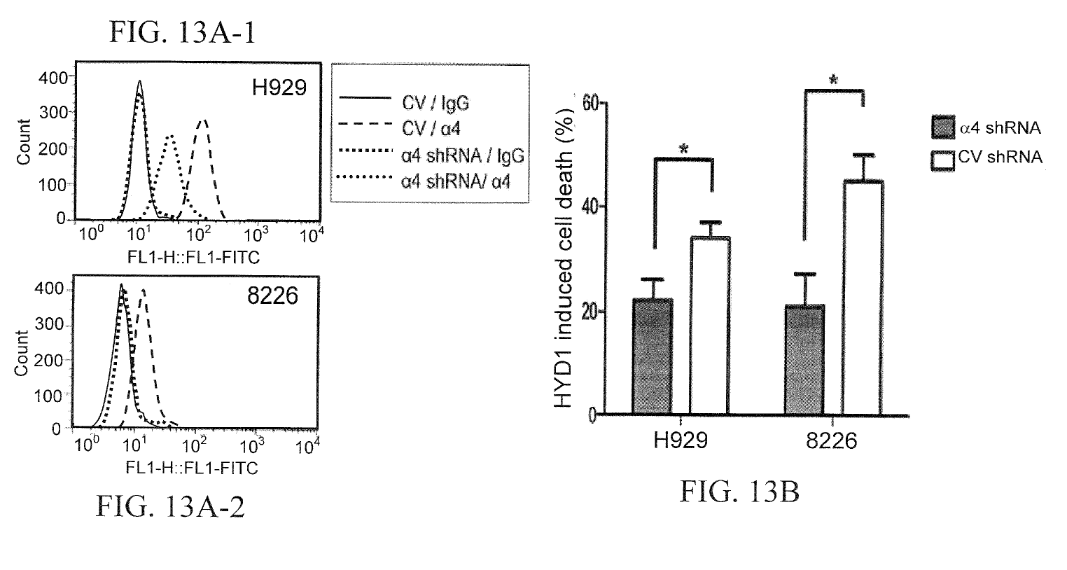

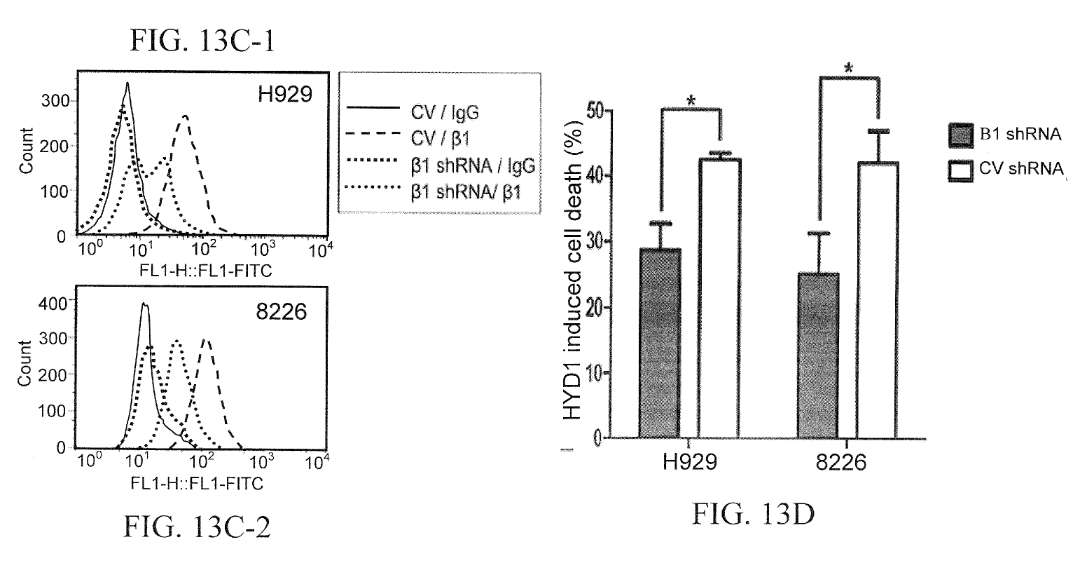

Considering H929-60 cells were resistant to HYD1 induced cell death and demonstrate reduced surface expression of the α4 integrin subunit, the inventors next determined whether reducing the expression of α4 integrins using shRNA targeting strategies in myeloma cell lines was sufficient to induce resistance towards HYD1 induced cell death. As shown in FIGS. 13A-1, 13A-2, and 13B, reducing the expression of α4 integrin conferred resistance to HYD1 (p<0.05, Student's t-test) in both 8226 and H929 cells. Since α4 integrin can heterodimerize with either β1 or β7 integrin, the inventors tested whether reducing β1 integrin was sufficient to induce resistance to myeloma cell lines. As shown in FIGS. 13C, 13C-1, 13C-2, and 13D, reducing β1 integrins rendered H929 and 8226 cells resistant to HYD1 induced cell death (p<0.05, student's t-test). β1 integrin can heterodimerize with 11 different a sub-units. The observation that reducing α4 or β1 integrin gave similar levels of protection indicates that the α4β1 integrin is the predominant β1 integrin partner associated with HYD1 induced cell death in myeloma cells. The observation that reducing integrin expression afforded only partial resistance may be due to (a) residual levels of α4β1 integrin remaining on the cell surface or (b) α4β1 is a component of the binding complex required for HYD1 induced cell death.

### Example 11

### Biotin-HYD1 Interacts with α4 Integrin and Reduced Binding was Observed in the Acquired Drug Resistant Cell Line

Previous data indicated that biotin-HYD1 associated with α3 and α6 on prostate cancer cell lines (15). The inventors utilized a similar strategy to determine whether biotin-HYD1 interacted with an α4 integrin containing complex and whether that interaction was attenuated in the resistant cell line. The total membrane lysate was used as a control for detection of α4 integrin. Again the reduction in the cleaved α4 integrin subunit in membrane extracts (although not as dramatic as the whole cell lysate), was most prominent in the resistant cell line compared to the mature α4 integrin subunit. Additionally, as shown in FIG. 14, using biotin-HYD1 the inventors demonstrate that the resistant cell line shows preferential decreased binding for the cleaved α4 subunit.

### Example 12

### H929-60 Cells Displayed Reduced Binding to HS-5 Bone Marrow Stromal Cells and Demonstrate a Compromised CAM-DR Phenotype

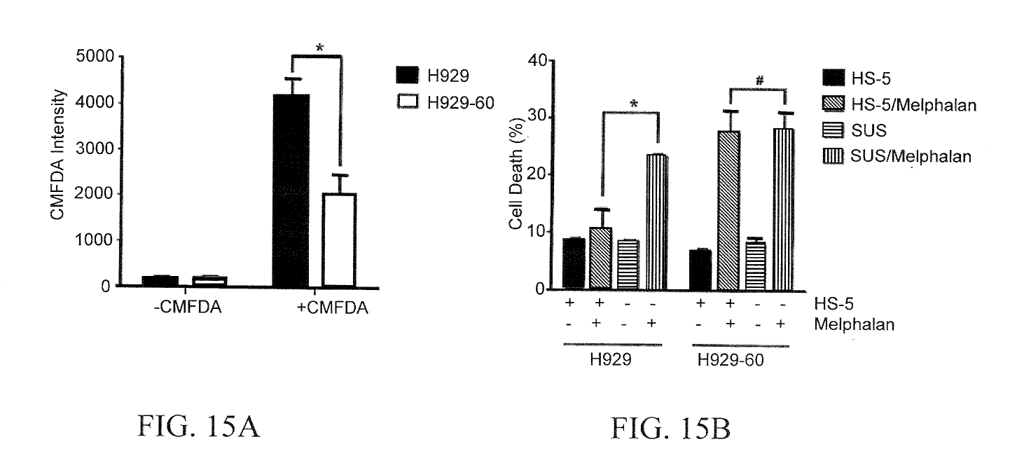

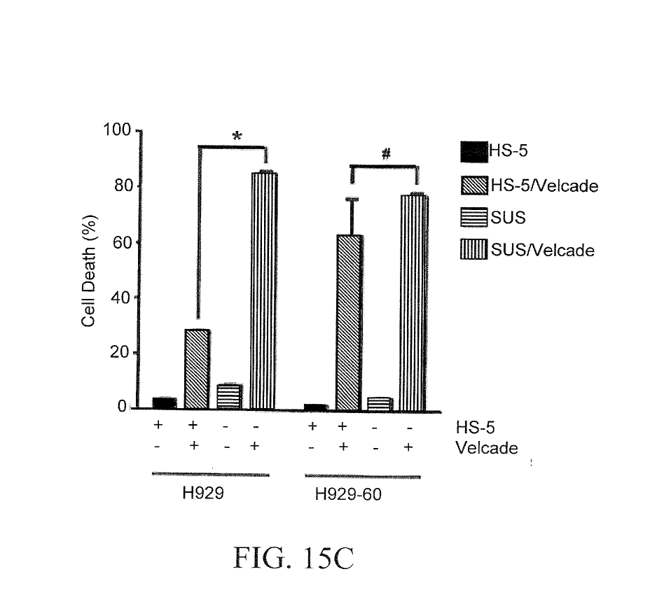

In addition to attenuated adhesion to FN and VCAM-1, H929-60 cells also exhibit a reduction in adhesion to HS-5 stromal cells (FIG. 15A). The inventors reasoned that acquisition of resistance towards HYD1, which correlated with reduced functional binding to fibronectin, VCAM-1, and HS-5 stromal cells, would likely result in a compromised CAMDR phenotype. To test this premise, the inventors utilized the HS-5 co-culture model of drug resistance. H929 and the HYD1 resistant variant H929-60 cells were treated with either melphalan or velcade in the presence or absence of HS-5/GFP bone marrow stromal cells. As shown in FIGS. 15B and 15C, the HYD1 resistant cell line was not resistant in the co-culture bone marrow stroma model to melphalan or velcade, respectively. Together, these data indicate that as myeloma cells are selected for resistance to HYD1 they are losing resistance to standard chemotherapy in the context of the bone marrow microenvironment due to reduced capacity to adhere to bone marrow stroma cells. Again, these data support the premise that disrupting adhesive interactions are likely to improve the efficacy of standard chemotherapy in the context of the bone marrow microenvironment.

### Example 13

### HYD1 Induced Cell Death is Increased in Relapsed Myeloma Patient Specimens Compared to Newly Diagnosed Specimens and Correlates with α4 Integrin Expression

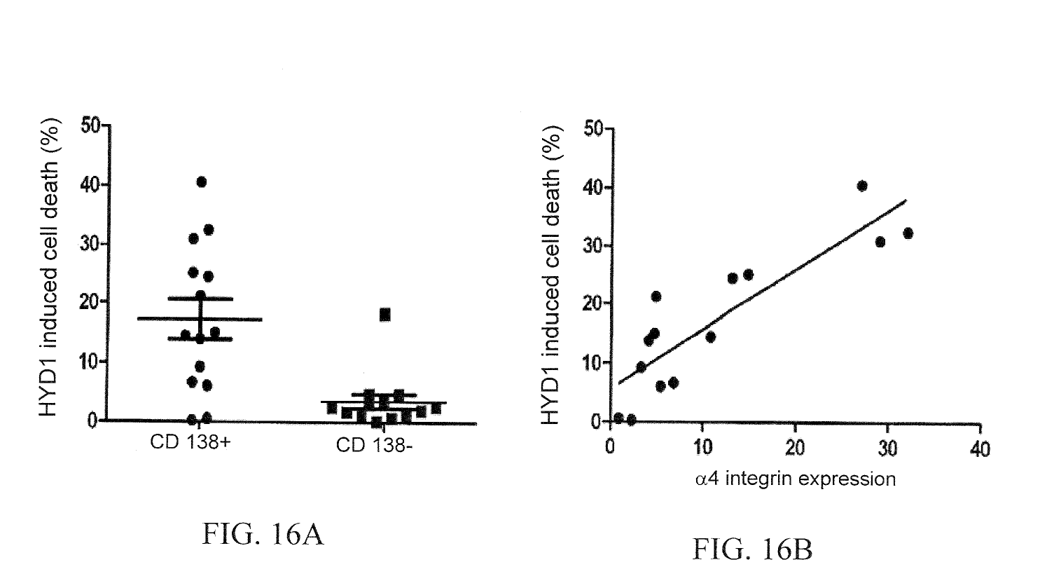

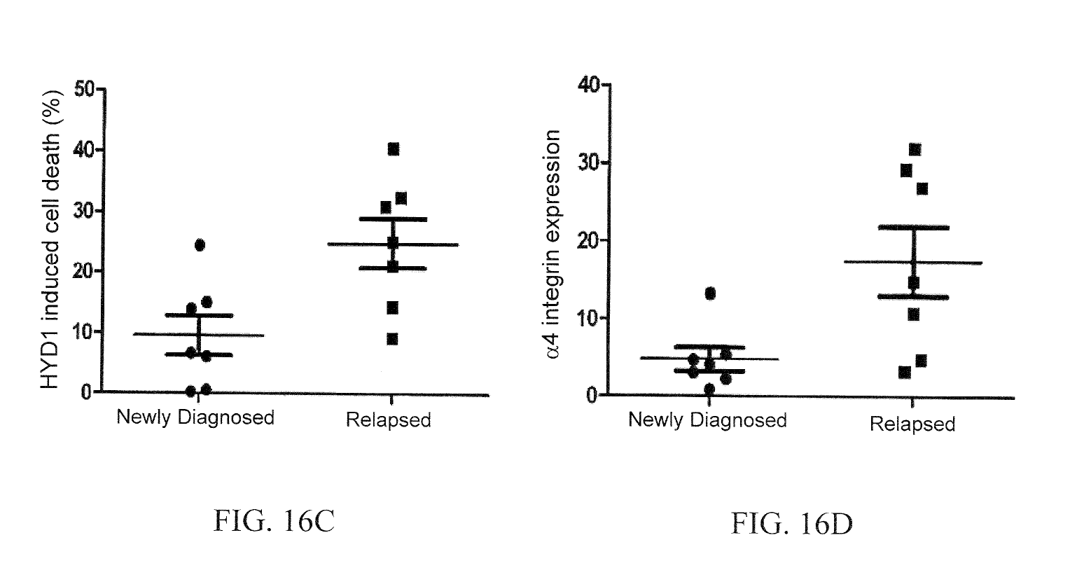

In order to determine whether primary myeloma specimens are sensitive to HYD1 induced cell death, the inventors collected 7 newly diagnosed and 7 relapsed primary myeloma specimens. Immunomagnetic beads were used to enrich for CD138 positive malignant plasma cell fractions. As shown in FIG. 16A, the CD138 positive tumor population was more sensitive to HYD1 induced cell death compared to the CD138 negative population. In addition to measuring cell death, the inventors determined by FACS analysis the levels of α4 integrin expression in the CD138 positive cells. As shown in FIG. 16B, HYD1 induced cell death positively correlates with α4 integrin expression. Importantly, the inventors observed that HYD1 was significantly (p<0.05, student's t-test) more active in relapsed refractory patients compared to newly diagnosed patients (See FIG. 16C). Finally as shown in FIG. 16D, the inventors show that α4 integrin levels are increased in CD138 positive cells isolated from relapsed myeloma patients compared to newly diagnosed patients (p<0.05, student's t-test). Together, these data suggest that α4 integrin expression is selected for over the course of drug treatment and may contribute to the eventual failure to therapeutically manage multiple myeloma. Additionally, patients with high levels of α4 expression may benefit from combination strategies that include targeting this specific integrin complex such as Natalizumab (21) (humanized α4 antibody) or HYD1.

### Example 14

### The Forced Interaction of α4 Integrin and CD44 by the Novel Peptide HYD1 May be a Determinant of HYD1-Induced Cell Death in Multiple Myeloma

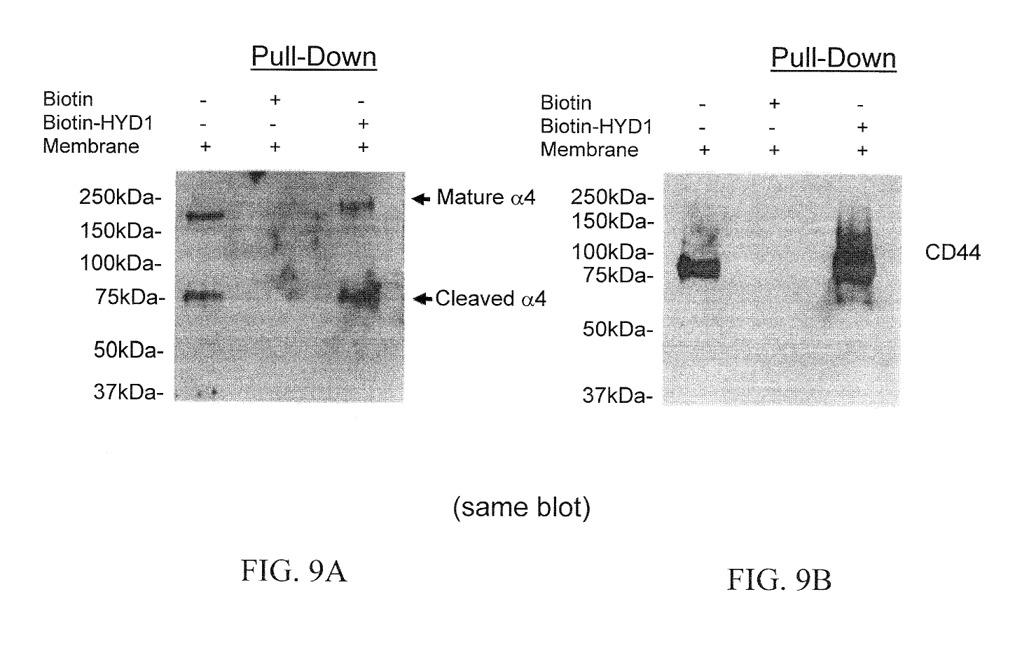

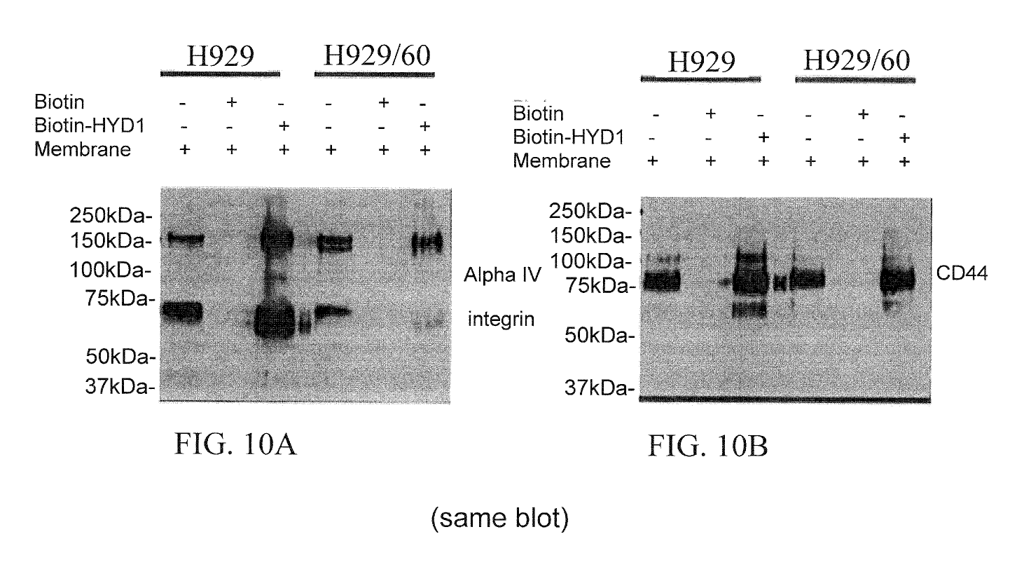

The inventors' laboratory has shown that treatment with the d-amino acid peptide HYD1 (KIKMVISWKG; SEQ ID NO:1) induces necrotic cell death in multiple myeloma (MM) cell lines. Furthermore, the inventors have implicated the adhesion receptor VLA-4 as being involved in this process via shRNA silencing strategies. However, reducing α4 integrin only demonstrated partial protection indicating that other components of the HYD1 binding complex may be required for cell death. Thus, the inventors investigated the possible role of other adhesion molecules and/or complexes that may contribute to HYD1-induced cell death. Using an unbiased approach, the inventors performed a total membrane pull-down with biotin conjugated HYD1 and NeutrAvidin beads on NCI-H929 MM cells. Samples were then loaded on an SOS-PAGE gel and analyzed by Mass Spectrometry. Data were then mined with the Scaffold 3 proteome software to determine pertinent hits. The adhesion receptor CD44 was determined to have the most peptide match hits and was confirmed as a valid hit via Western Blot analysis. Preliminary data using ELISA based binding studies with biotin-HYD1 as bait showed that recombinant CD44 directly binds to biotin-HYD1 in a concentration dependent manner. In order to identify complexes involved in HYD1-induced cell death, membrane immunoprecipitation assays were performed using CD44 and Integrin α4 mAbs in both the presence and absence of HYD1. While Western Blot analysis confirmed basal levels of complex interactions between α4 integrin and CD44, the complex was significantly enhanced in the presence of HYD1. These data not only indicate a possible role of CD44 in HYD1-induced cell death, but also the ability of HYD1 to induce interactions among adhesion molecules and complexes. Results are shown in FIGS. 9A, 9B, 10A, and 10B.

The development of an isogenic resistant cell line has been used by many investigators as model systems for rapidly delineating important molecular determinants of response for novel as well as clinically approved agents (22-26). Historically, some of these cell line models have allowed for the identification of targets associated with drug sensitivity and resistance (27). Selection with HYD1 resulted in a cell line with compromised adhesive interactions towards extracellular matrices and HS-5 bone marrow stroma cells. The inventors reasoned that a reduction in functional binding to the HS-5 bone marrow stroma cell line may render the HYD1 resistant cell line sensitive to standard therapy in the co-culture model of drug resistance. Indeed this was found to be true, as the HYD1 resistant cell line was not resistant to melphalan or velcade induced cell death in the co-culture model of drug resistance. Moreover, the data herein demonstrate that α4 integrin expression and sensitivity towards HYD1 was increased in specimens obtained from relapsed patients compared to newly diagnosed patients, suggesting that α4 expression is selected for either as a consequence of standard drug exposure or disease progression.

Acquisition of resistance towards melphalan and doxorubicin in myeloma cells lines as expression of α4 integrin increased with selection for drug resistance (6). Agents that disrupt cell adhesion may indeed be a good strategy for assessing the overall fitness of cells in the context of the bone marrow microenvironment. Thus, agents that disrupt cell adhesion may be important to test the development of evolutionary based double bind strategies, where resistance to a drug is predicted to occur at the cost of fitness within the niche. The utilization of double bind strategies is a unique concept recently proposed by Gatenby and colleagues for delaying the emergence of resistant variants in the treatment of cancer and indeed HYD1 may fit the criteria for an agent to test this unique therapeutic strategy (28). The data herein indicate that reducing α4β1 integrin expression was sufficient to confer partial drug resistance. However, since only partial resistance was observed, additional components contained within the HYD1 binding complex may contribute to HYD1-induced cell death. Further studies may be conducted for delineating the entire HYD1 interacting complex. The inventors were able to show that expression of α4 integrin in primary patient specimens correlated with sensitivity towards HYD1-induced cell death ex-vivo, a finding that correlated well with the inventors' cell line observations. The inventors propose that to ensure adequate designs of trials, it is essential to define biomarkers of response using patient specimens in early phases of drug development, as response markers can take time to validate and often lag behind the design of early clinical trials (29). Additionally, the inventors' studies indicate that a larger study determining the prognostic value of VLA-4 integrin is warranted in multiple myeloma. In the drug resistant variant cell line, the inventors observed a predominant reduction in the cleavage of α4 integrin compared to the mature form. Recent data demonstrate that targeting α4 integrin in a syngeneic mouse 5TGM1 model via monoclonal antibody treatment reduced the tumor burden in the bone marrow, spleen and liver (30). Moreover, the VCAM-1/VLA-4 axis increases MIP-1 alpha and beta levels and increases the ability of myeloma cells to support osteoclastogenesis (31). Based on these findings, it will be informative to determine whether HYD1 inhibits the ability of myeloma cells to disrupt bone homeostasis by either inhibiting the activation of osteoclasts or disrupting the ability of myeloma cells to inhibit the differentiation of osteoblasts (32-34). Another strategy for targeting integrins is to inhibit pathways required for inside-out activation of VLA-4 integrins. Thus, VLA-4 can be modulated by regulating the affinity for ligand as well as clustering or avidity of the integrin heterodimer (35, 36). As a result, feasible strategies could include targeting Rap1 which is known to be required for inside out activation of VLA-4 (37). However, HYD1 is unique, to the inventors' knowledge, in that in addition to blocking cell adhesion, HYD1 induces necrotic cell death directly on the myeloma cell, a finding, that was not observed with α4 blocking antibody or RGD containing peptides (data not shown).

Historically, drug development has focused on aberrations in signaling intrinsic to the tumor cells using unicellular models. However, it has been argued that tumors evolve in the context of the microenvironment, and thus it is feasible that some of the phenotypes observed in tumors such as drug resistance and metastasis will only be expressed in the context of cues derived from the microenvironment (38-41). In summary, the data herein support the premise that targeting survival signals that occur between tumor cells and the microenvironment is an attractive strategy for increasing the therapeutic potential of treatment regimens (particularly, combination regimens) and may lead to better clinical management of cancers such as multiple myeloma considered to be intrinsically resistant to standard therapy with no currently available curative strategies.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Anderson K C, Kyle R A, Dalton W S, Landowski T, Shain K, Jove R, et al. Multiple Myeloma: New Insights and Therapeutic Approaches. Hematology (Am Soc Hematol Educ Program) 2000: 147-165.
2. Kyle R A, Rajkumar S V. Multiple myeloma. N Engl J Med 2004 Oct. 28; 351(18):1860-1873.
3. Jemalk A, Siegel R, Xu J, Ward E. Cancer Statistics, 2010. CA Cancer J Clin Ju17.
4. Aoudjit F, Vuori K. Integrin signaling inhibits paclitaxel-induced apoptosis in breast cancer cells. Oncogene 2001 Aug. 16; 20(36): 4995-5004.
5. Azab A K, Runnels J M, Pitsillides C, Moreau A S, Azab F, Leleu X, et al. The CXCR4 inhibitor AMD3100 disrupts the interaction of multiple myeloma cells with the bone marrow microenvironment and enhances their sensitivity to therapy. Blood 2009 Jan. 12.
6. Damiano J S, Cress A E, Hazlehurst L A, Shtil A A, Dalton W S. Cell adhesion mediated drug resistance (CAM-DR): role of integrins and resistance to apoptosis in human myeloma cell lines. Blood 1999 Mar. 1; 93(5): 1658-1667.
7. Hazlehurst L A, Enkemann S A, Beam C A, Argilagos R F, Painter J, Shain K H, et al. Genotypic and phenotypic comparisons of de novo and acquired melphalan resistance in an isogenic multiple myeloma cell line model. Cancer Res 2003 Nov. 15; 63(22): 7900-7906.
8. Mori Y, Shimizu N, Dallas M, Niewolna M, Story B, Williams P J, et al. Antialpha4 integrin antibody suppresses the development of multiple myeloma and associated osteoclastic osteolysis. Blood 2004; 104(7): 2149.
9. Nefedova Y, Landowski T H, Dalton W S. Bone marrow stromal-derived soluble factors and direct cell contact contribute to de novo drug resistance of myeloma cells by distinct mechanisms. Leukemia 2003 June; 17(6): 1175-1182.
10. Nefedova Y, Sullivan D M, Bolick S C, Dalton W S, Gabrilovich D I. Inhibition of Notch signaling induces apoptosis of myeloma cells and enhances sensitivity to chemotherapy. Blood 2008 Feb. 15; 111(4): 2220-2229.
11. Sethi T, Rintoul R C, Moore S M, MacKinnon A C, Salter D, Choo C, et al. Extracellular matrix proteins protect small cell lung cancer cells against apoptosis: a mechanism for small cell lung cancer growth and drug resistance in vivo. Nat Med 1999 June; 5(6): 662-668.
12. Morin P J. Drug resistance and the microenvironment: nature and nurture. DrugResist Updat 2003 August; 6(4): 169-172.
13. Matsunaga T, Takemoto N, Sato T, Takimoto R, Tanaka I, Fujimi A, et al. Interaction between leukemic-cell VLA-4 and stromal fibronectin is a decisive factor for minimal residual disease of acute myelogenous leukemia. Nat Med 2003 September; 9(9): 1158-1165.
14. DeRoock I B, Pennington M E, Sroka T C, Lam K S, Bowden G T, Bair E L, et al. Synthetic peptides inhibit adhesion of human tumor cells to extracellular matrix proteins. Cancer Res 2001 Apr. 15; 61(8): 3308-3313.
15. Sroka T C, Pennington M E, Cress A E. Synthetic D-amino acid peptide inhibits tumor cell motility on laminin-5. Carcinogenesis 2006 Mar. 14.
16. Nair R R, Emmons M F, Cress A E, Argilagos R F, Lam K, Kerr W T, et al. HYD1- induced increase in reactive oxygen species leads to autophagy and necrotic cell death in multiple myeloma cells. Mol Cancer Ther 2009 August; 8(8): 2441-2451.
17. Hazlehurst L A, Valkov N, Wisner L, Storey J A, Boulware D, Sullivan D M, et al. Reduction in drug-induced DNA double-strand breaks associated with beta1 integrin-mediated adhesion correlates with drug resistance in U937 cells. Blood 2001 Sep. 15; 98(6): 1897-1903.
18. Hazlehurst L A, Foley N E, Gleason-Guzman M C, Hacker M P, Cress A E, Greenberger L W, et al. Multiple mechanisms confer drug resistance to mitoxantrone in the human 8226 myeloma cell line. Cancer Res 1999 Mar. 1; 59(5): 1021-1028.
19. Bednarczyk J L, Szabo M C, McIntyre B W. Post-translational processing of the leukocyte integrin alpha 4 beta 1. J Biol Chem 1992 Dec. 15; 267(35): 25274-25281.
20. Teixido J, Parker C M, Kassner P D, Hemler M E. Functional and structural analysis of VLA-4 integrin alpha 4 subunit cleavage. J Biol Chem 1992 Jan. 25; 267(3): 1786-1791.
21. Baker D E. Natalizumab: overview of its pharmacology and safety. Rev Gastroenterol Disord 2007 Winter; 7(1): 38-46.

22. Gutman D, Morales A A, Boise L H. Acquisition of a multidrug-resistant phenotype with a proteasome inhibitor in multiple myeloma. Leukemia 2009 November; 23(11): 2181-2183.
23. Banerjee D, Mayer-Kuckuk P, Capiaux G, Budak-Alpdogan T, Gorlick R, Bertino J R. Novel aspects of resistance to drugs targeted to dihydrofolate reductase and thymidylate synthase. Biochim Biophys Acta 2002 Jul. 18; 1587(2-3): 164-173.
24. Mahon F X, Deininger M W, Schultheis B, Chabrol J, Reiffers J, Goldman J M, et al. Selection and characterization of BCR-ABL positive cell lines with differential sensitivity to the tyrosine kinase inhibitor STI571: diverse mechanisms of resistance. Blood 2000 Aug. 1, 96(3): 1070-1079.
25. Morgan S E, Kim R, Wang P C, Bhat U G, Kusumoto H, Lu T, et al. Differences in mutant p53 protein stability and functional activity in teniposide-sensitive and -resistant human leukemic CEM cells. Oncogene 2000 Oct. 12; 19(43): 5010-5019.
26. Robey R W, Polgar O, Deeken J, To K W, Bates S E. ABCG2: determining its relevance in clinical drug resistance. Cancer Metastasis Rev 2007 March; 26(1): 39-57.
27. Gottesman M M, Ling V. The molecular basis of multidrug resistance in cancer: the early years of P-glycoprotein research. FEBS Lett 2006 Feb. 13; 580(4): 998-1009.
28. Gatenby R A, Silva A S, Gillies R J, Frieden B R. Adaptive therapy. Cancer Res 2009 Jun. 1; 69(11): 4894-4903.
29. Khleif S N, Doroshow J H, Hait W N. AACR-FDA-NCI Cancer Biomarkers Collaborative consensus report: advancing the use of biomarkers in cancer drug development. Clin Cancer Res July 1; 16(13): 3299-3318.
30. Olson D L, Burkly L C, Leone D R, Dolinski B M, Lobb R R. Anti-alpha4 integrin monoclonal antibody inhibits multiple myeloma growth in a murine model. Mol Cancer Ther 2005 January; 4(1): 91-99.
31. Abe M, Hiura K, Ozaki S, Kido S, Matsumoto T. Vicious cycle between myeloma cell binding to bone marrow stromal cells via VLA-4-VCAM-1 adhesion and macrophage inflammatory protein-1alpha and MIP-1beta production. J Bone Miner Metab 2009; 27(1): 16-23.
32. Epstein J, Yaccoby S. Consequences of interactions between the bone marrow stroma and myeloma. Hematol J2003; 4(5): 310-314.
33. Tian E, Zhan F, Walker R, Rasmussen E, Ma Y, Barlogie B, et al. The role of the Wnt-signaling antagonist DKK1 in the development of osteolytic lesions in multiple myeloma. N Engl J Med 2003 Dec. 25; 349(26): 2483-2494.
34. Yaccoby S, Wezeman M J, Henderson A, Cottler-Fox M, Yi Q, Barlogie B, et al. Cancer and the microenvironment: myeloma-osteoclast interactions as a model. Cancer Res 2004 Mar. 15; 64(6): 2016-2023.
35. Abram C L, Lowell C A. The ins and outs of leukocyte integrin signaling. Annu Rev Immunol 2009; 27: 339-362.
36. Luo B H, Calinan C V, Springer T A. Structural basis of integrin regulation and signaling. Annu Rev Immunol 2007; 25: 619-647.
37. de Bruyn K M, Rangarajan S, Reedquist K A, Figdor C G, Bos J L. The small GTPase Rap1 is required for Mn(2+)- and antibody-induced LFA-1- and VLA-4-mediated cell adhesion. J Biol Chem 2002 Aug. 16; 277(33): 29468-29476.
38. Bissell M J, Radisky D. Putting tumours in context. Nat Rev Cancer 2001 October; 1(1): 46-54.
39. Hazlehurst L A, Landowski T H, Dalton W S. Role of the tumor microenvironment in mediating de novo resistance to drugs and physiological mediators of cell death. Oncogene 2003 Oct. 20; 22(47): 7396-7402.
40. McMillin D W, Delmore J, Weisberg E, Negri J M, Geer D C, Klippel S, et al. Tumor cell-specific bioluminescence platform to identify stroma-induced changes to anticancer drug activity. Nat Med April; 16(4): 483-489.
41. Nair R R, Tolentino J, Hazlehurst L A. The bone marrow microenvironment as a sanctuary for minimal residual disease in CML. Biochem Pharmacol September 1; 80(5): 602-612.

1. A method for treating a malignancy in a subject, comprising administering:
(a) an effective amount of an agent that binds CD44 to the subject, wherein the malignancy has at least one of the following characteristics: the malignancy is a relapsing malignancy, the malignancy is one with elevated expression or activity of the α4 integrin subunit, and the malignancy expresses CD44; or
(b) an effective amount of an agent that binds CD44 to the subject, and an effective amount of an agent that increases the expression or activity of the α4 integrin subunit in cells of the malignancy (malignant cells); or
(c) an agent that binds CD44, and at least one anti-cancer agent to the subject.

2. The method of claim 1, wherein the CD44 binding agent comprises or consists of a HYD1 peptide.

3. The method of claim 1, wherein the method comprises (a), and wherein the malignancy exhibits elevated expression or activity of the α4 integrin.

4. The method of claim 1, wherein the method comprises (a), and wherein the malignancy expresses CD44.

5. The method of claim 1, wherein the method comprises (a), and wherein the malignancy exhibits elevated expression or activity of the α4 integrin, and the malignancy expresses CD44.

6. The method of claim 1, wherein the method comprises (a), and wherein the malignancy is a relapsing malignancy.

7-9. (canceled)

10. A method for inhibiting the growth of a cancer cell in vitro or in vivo, comprising administering:
(a) an effective amount of an agent that binds CD44 to the cell in vitro or in vivo to inhibit cell growth, wherein the cancer cell has at least one of the following characteristics: the cancer cell is that of a relapsing cancer, the cancer cell expresses CD44, and/or the cancer cell is one with elevated expression or activity of the α4 integrin subunit; or
(b) an effective amount of an agent that binds CD44 and an effective amount of an agent that increases the expression or activity of the α4 integrin subunit to the cell in vitro or in vivo to inhibit cell growth.

11. The method of claim 10, wherein the CD44 binding agent comprises or consists of a HYD1 peptide.

12-13. (canceled)

14. A method for selecting agents that can enhance the cytotoxic response of a cancer cell to an agent that binds CD44, comprising selecting an agent that is predetermined to be effective in increasing the expression or activity of the α4 integrin subunit.

15. The method of claim 14, wherein the CD44 binding agent comprises or consists of a HYD1 peptide.

16. The method of claim 14, wherein the method comprises determining whether a candidate agent increases the expression or activity of the α4 integrin subunit in a cancer cell in vitro or in vivo, and selecting the candidate agent for treatment if the candidate agent increases the expression or activity of the α4 integrin subunit in the cancer cell in vitro or in vivo.

17-18. (canceled)

19. A method for determining whether a cancer will be sensitive or resistant to treatment with an agent that binds CD44, comprising assessing one or more of the following parameters in a cell sample of the cancer: expression or activity of the α4 integrin subunit, CD44 expression, CD138 expression, functional binding to fibronectin, functional binding to VCAM-1, and functional binding to HS-5 stromal cells; wherein one or more of reduced expression or activity of the α4 integrin subunit, reduced functional binding to fibronectin, reduced functional binding to VCAM-1, lack of CD44 expression, lack of CD138 expression, and reduced functional binding to HS-5 stromal cells are indicative of resistance or lack of sensitivity; and wherein one or more of elevated expression or activity of the α4 integrin subunit, CD44 expression, CD138 expression, elevated functional binding to fibronectin, elevated functional binding to VCAM-1, and elevated functional binding to HS-5 stromal cells are indicative of sensitivity or lack of resistance.

20. The method of claim 19, wherein the CD44 binding agent comprises or consists of a HYD1 peptide.

21-41. (canceled)

42. A composition, comprising:
(a) an agent that binds CD44; and an additional agent that increases the expression or activity of the α4 integrin subunit in a malignancy; or
(b) an agent that binds CD44; and an additional agent that decreases the expression or activity of the α4 integrin subunit in a malignancy; or
(c) an agent that binds CD44; and at least one additional agent selected from among suberoylanilide hydroxamic acid (SAHA) or other histone deacetylase inhibitor, arsenic trioxide, doxorubicin or other anthracycline DNA intercalating agent, and etoposide or other topoisomerase II inhibitor; or
(d) an array comprising a substrate and two or more capture probes disposed thereon, wherein said two or more capture probes comprise or consist of:
(i) antibodies, or antibody fragments, that specifically bind alpha4 integrin and CD44; or
(ii) oligonucleotides that are partially or fully complementary to, and bind to, nucleic acid sequences encoding alpha4 integrin and CD44; or
(e) a cell line exhibiting resistance to HYD1 peptide-induced cell death, and isolated cells there from.

43. The composition of claim 42, wherein the CD44 binding agent comprises or consists of a HYD1 peptide.

44-49. (canceled)

50. The composition of claim 42, wherein the composition comprises (c), and wherein the CD44 binding agent comprises or consists of a HYD1 peptide.

51-54. (canceled)

55. The composition of claim 42, wherein the composition comprises (e), and wherein the cell line is a variant H929 cell line.

56. (canceled)

57. A method for producing a cell line with resistance to HYD1 peptide-induced cell death, comprising culturing a sensitive cell in the presence of increasing amounts of a HYD1 peptide for a period of time sufficient to produce a cell with resistance to HYD1 peptide-induced cell death.

58-59. (canceled)

60. The composition of claim 42, wherein the composition comprises (d), and wherein the array further comprises one or more capture probes comprising or consisting of:
antibodies, or antibody fragments, that specifically bind a tumor-specific or tumor-associated antigen; or
oligonucleotides that are partially or fully complementary to, and bind to, nucleic acid sequences encoding a tumor-specific or tumor-associated antigen.

61. The composition of claim 60, wherein the tumor-specific or tumor-associated antigen is selected from among CD 138, CD34, cytokeratin 7, or a tumor marker listed in Table 1.

62-64. (canceled)
